(19) United States
(12) Patent Application Publication
(10) Pub. No.: US 2011/0306572 A1
(43) Pub. Date: Dec. 15, 2011

(54) COMBINATION THERAPY FOR THE TREATMENT OF CANCER USING COX-2 INHIBITORS AND DUAL InHIBITORS OF EGFR [ErbB1] AND HER-2 [ErbB2]

(75) Inventors: Thomas M. Estok, Carlsbad, CA (US); Sara L. Zaknoen, Carlsbad, CA (US); Robert K. Mansfield, Carlsbad, CA (US)

(73) Assignee: TRAGARA PHARMACEUTICALS, INC, San Diego, CA (US)

(21) Appl. No.: 13/130,550

(22) PCT Filed: Sep. 23, 2008
(86) PCT No.: PCT/US08/77402

§ 371 Date: Aug. 23, 2011

Related U.S. Application Data
(60) Provisional application No. 60/974,731, filed on Sep. 24, 2007.

(51) Int. Cl. A61K 31/517; A61K 31/7068; A61P 35/00

(52) U.S. Cl. 514/49; 514/266.24; 514/266.2

(57) ABSTRACT

Described herein are compositions and methods for using these compositions in the treatment of cancer, tumors, and tumor-related disorders in a subject.

### CROSS-REFERENCE

This application claims the benefit of U.S. Provisional Application No. 60/974,731, filed Sep. 24, 2007, which is incorporated herein by reference in its entirety.

### FIELD

The present invention relates to combination compositions and the use of such combinations for the treatment of cancer, tumors, and tumor-related disorders.

### BACKGROUND

Cancer, tumors, tumor-related disorders, and neoplastic disease states are serious and often times life-threatening conditions. These diseases and disorders, which are generally characterized by rapidly-proliferating cell growth, continue to be the subject of research efforts directed toward the identification of therapeutic agents which are effective in the treatment thereof. Such agents may prolong the survival of the patient, inhibit the rapidly-proliferating cell growth associated with the neoplasm, or effect a regression of the neoplasm.

Generally, surgery and radiation therapy are the first modalities considered for the treatment of cancer that is considered locally confined, and offer the best prognosis. Chemotherapy treatment of certain cancers typically results in disappointing survival rates but still offers a survival benefit. For example, in patients with advanced metastatic breast cancer, a chemotherapy regimen, such as the use of paclitaxel, docetaxel, anthracycline, or trastuzumab is employed. If patients do not respond to this therapy, a course of treatment involving lapatinib in combination with capecitabine can be followed. Lapatinib targets both the epidermal growth factor receptor {EGFR [ErbB1]) tyrosine kinase and the HER2 [ErbB2] receptor tyrosine kinase which are highly expressed and occasionally mutated in various forms of cancer. If patients fail to respond to lapatinib treatment, additional conventional treatment offers limited benefit.

Despite lapatinib's approval for the treatment of advanced metastatic breast cancer, as with most therapeutic agents, side-effects result from its use. For example, common side effects, occurring in greater than 20% of patients taking lapatinib include, rash, diarrhea, fatigue, palmar plantar erythrodysesthesia, nausea and vomiting. Additionally, less common side effects include decrease in left ventricular ejection fraction and QT prolongation. Of greater concern, is the growing view that, while utilization of lapatinib for the treatment of tumors may initially shrink the size of the tumor, the tumor may eventually enlarge in size, indicating, among other things, the development of resistance. Lapatinib may be representative of the types of therapeutic agents being used for cancer treatment; in that its use has an effect on cancer, but because of other factors, which are not entirely known, the tumor develops resistance and progresses.

What is needed, therefore, are compositions and/or methods of treatment for cancer which take advantage of the synergy found in a therapeutic combination that could increase the effectiveness of the agents and reduce and/or eliminate the side effects typically associated with conventional treatments.

### SUMMARY OF THE INVENTION

Provided herein are methods of treating cancer based on the administration of a combination of a 1,2-diphenylpyrrole derivative (a COX-2 selective inhibitor) and an inhibitor of both EGFR [ErbB1] and HER2 [ErbB2]. The methods may further include treatments wherein the combination is supplemented with one or more therapeutic agents or therapies. In one method, capecitabine is administered in combination with the 1,2-diphenylpyrrole and the inhibitor of both EGFR [ErbB1] and HER2 [ErbB2]. The latter combination is useful in the treatment of breast cancers associated with overexpression of HER2 [ErbB2]. The 1,2-diphenylpyrrole derivative and the inhibitor of both EGFR [ErbB1] and HER2 [ErbB2] may be provided in separate dosage forms or combined in one dosage form (e.g. a fixed dose).

In one embodiment, the invention provides a composition comprising a combination of a 1,2-diphenylpyrrole derivative and an inhibitor of both EGFR [ErbB1] and HER2 [ErbB2] wherein the NSAID-induced side effects are substantially diminished. For example, NSAID-induced side effects include, but are not limited to, nausea, vomiting, diarrhea, constipation, abdominal pain, gastritis, duodenitis, gastric bleeding, duodenal bleeding, decreased appetite, rash, dizziness, headache, drowsiness, fluid retention, edema, kidney failure, liver failure, ulcers and prolonged bleeding after surgery. In another embodiment, the invention provides a composition comprising a combination of a 1,2-diphenylpyrrole derivative and an inhibitor of both EGFR [ErbB1] and HER2 [ErbB2] wherein the 1,2-diphenylpyrrole derivative is 2-(4-ethoxyphenyl)-4-methyl-1-(4-sulfamoylphenyl)-pyrrole and the inhibitor of both EGFR [ErbB1] and HER2 [ErbB2] is lapatinib and wherein the NSAID-induced side effects are substantially diminished.

1,2-Diphenylpyrrole derivatives described herein have the general formula:

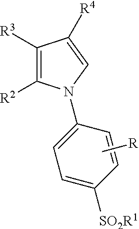

wherein:

R is a hydrogen atom, a halogen atom or an alkyl group having from 1 to 6 carbon atoms;

R¹ is an alkyl group having from 1 to 6 carbon atoms or an amino group;

R² is a phenyl group which is unsubstituted or is substituted by at least one substituent selected from the group consisting of substituents α and substituents β;

R³ is a hydrogen atom, a halogen atom or an alkyl group which has from 1 to 6 carbon atoms and which is unsubstituted or is substituted by at least one substituent selected from the group consisting of a hydroxy group, a halogen atom, an alkoxy group having from 1 to 6 carbon atoms and an alkylthio group having from 1 to 6 carbon atoms;

R⁴ is a hydrogen atom; an alkyl group which has from 1 to 6 carbon atoms and which is unsubstituted or is substituted by at least one substituent selected from the group consisting of a hydroxy group, a halogen atom, an alkoxy group having from 1 to 6 carbon atoms and an alkylthio group having from 1 to 6 carbon atoms; a cycloalkyl group having from 3 to 8 carbon atoms, an aryl group; or an aralkyl group; said aryl group having from 6 to 14 ring carbon atoms in a carbocyclic ring and are unsubstituted or are substituted by at least one substituent selected from the group consisting of substituents α and substituents β; said aralkyl group are an alkyl group having from 1 to 6 carbon atoms and which are substituted by at least one aryl group as defined above;

said substituents α are selected from the group consisting of a hydroxy group, a halogen atom, an alkoxy group having from 1 to 6 carbon atoms and an alkylthio group having from 1 to 6 carbon atoms;

said substituents β are selected from the group consisting of an alkyl group which has from 1 to 6 carbon atoms and which is unsubstituted or are substituted by at least one substituent selected from the group consisting of a hydroxy group, a halogen atom, an alkoxy group having from 1 to 6 carbon atoms and an alkylthio group having from 1 to 6 carbon atoms; an alkanoyloxy group having from 1 to 6 carbon atoms; a mercapto group; an alkanoylthio group having from 1 to 6 carbon atoms; an alkylsulfinyl group having from 1 to 6 carbon atoms; a cycloalkloxy group having from 3 to 8 carbon atoms; a haloalkoxy group having from 1 to 6 carbon atoms; and an alkylenedioxy group having from 1 to 6 carbon atoms; or a pharmaceutically acceptable salt, solvate, or prodrug.

In one embodiment, the invention provides a 1,2-diphenylpyrrole derivative having the formula:

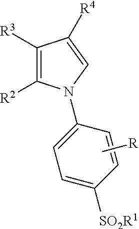

wherein:

R is a hydrogen atom, a halogen atom or an alkyl group having from 1 to 4 carbon atoms;

R¹ is a methyl group or an amino group;

R² is an unsubstituted phenyl group or a phenyl group which is substituted by at least one substituent selected from the group consisting of a halogen atom; an alkoxy group having from 1 to 4 carbon atoms; an alkylthio group having from 1 to 4 carbon atoms; an unsubstituted alkyl group having from 1 to 4 carbon atoms; an alkyl group having from 1 to 4 carbon atoms and which is substituted by at least one substituent selected from the group consisting of a halogen atom, an alkoxy group having from 1 to 4 carbon atoms and an alkylthio group having from 1 to 4 carbon atoms; a haloalkoxy group having from 1 to 4 carbon atoms; and an alkylenedioxy group having from 1 to 4 carbon atoms;

R³ is a hydrogen atom, a halogen atom, an unsubstituted alkyl group having from 1 to 4 carbon atoms or a substituted alkyl group having from 1 to 4 carbon atoms and substituted by at least one substituent selected from the group consisting of a halogen atom, an alkoxy group having from 1 to 4 carbon atoms and an alkylthio group having from 1 to 4 carbon atoms;

R⁴ is a hydrogen atom; an unsubstituted alkyl group having from 1 to 4 carbon atoms; a substituted alkyl group having from 1 to 4 carbon atoms and substituted by at least one substituent selected from the group consisting of a hydroxy group, a halogen atom, an alkoxy group having from 1 to 4 carbon atoms and an alkylthio group having from 1 to carbon atoms; a cycloalkyl group having from 3 to 6 carbon atoms; an aryl group which has from 6 to 10 ring carbon atoms and which is unsubstituted or is substituted by at least one substituent selected from the group consisting of a halogen atom; an alkoxy group having from 1 to 4 carbon atoms; an alkylthio group having from 1 to 4 carbon atoms; an unsubstituted alkyl group having from 1 to 4 carbon atoms; an alkyl group having from 1 to 4 carbon atoms and substituted by at least one substituent selected from the group consisting of a hydroxy group, a halogen atom, an alkoxy group having from 1 to 4 carbon atoms and an alkylthio group having from 1 to 4 carbon atoms; and a cycloalkyloxy group having from 3 to 7 carbon atoms; an aralkyl group having from 1 to 4 carbon atoms in the alkyl part and containing at least one said aryl group; or a pharmaceutically acceptable salt, solvate, or prodrug.

In one embodiment, the invention provides a 1,2-diphenylpyrrole derivative wherein:

R is a hydrogen atom;

R¹ is an amino group;

R² is an unsubstituted phenyl group or a phenyl group which is substituted by at least one substituent selected from the group consisting of a halogen atom, an alkoxy group having from 1 to 4 carbon atoms, an alkylthio group having from 1 to 4 carbon atoms, an alkyl group having from 1 to 4 carbon atoms, a haloalkyl group having from 1 to 4 carbon atoms, a haloalkoxy group having from 1 to 4 carbon atoms and a alkylenedioxy group having from 1 to 4 carbon atoms;

R³ is a hydrogen atom, a halogen atom, an alkyl group having from 1 to 4 carbon atoms or a haloalkyl group having from 1 to 4 carbon atoms;

R⁴ is a hydrogen atom; an unsubstituted alkyl group having from 1 to 4 carbon atoms; a substituted alkyl group having from 1 to 4 carbon atoms and substituted by at least one substituent selected from the group consisting of a hydroxy group and an alkoxy group having from 1 to 4 carbon atoms; a cycloalkyl group having from 3 to 6 carbon atoms; an aryl group which has from 6 to 10 ring carbon atoms and which is unsubstituted or is substituted by at least one substituent selected from the group consisting of a hydroxy group; a halogen atom; an alkoxy group having from 1 to 4 carbon atoms; an unsubstituted alkyl group having from 1 to 4 carbon atoms; an alkyl group having from 1 to 4 carbon atoms and which is unsubstituted or substituted by at least one halogen atom, and a cycloalkyloxy group having from 3 to 7 carbon atoms; and an aralkyl group having from 1 to 4 carbon atoms in the alkyl part and containing at least one said aryl group; or a pharmaceutically acceptable salt, solvate, or prodrug.

In one embodiment, R is a hydrogen atom. In another embodiment, R is a fluorine atom. In a further embodiment, R is a chlorine atom. In yet a further embodiment, R is a methyl group.

In one embodiment, R¹ is a methyl group. In another embodiment, R¹ is an amino group.

In one embodiment, R² is a phenyl group.

In one embodiment, R³ is a hydrogen atom. In another embodiment, R³ is a halogen atom.

In one embodiment, R⁴ is a hydrogen atom.

The term “aryl” refers to a carbocyclic aromatic hydrocarbon group having from 6 to 14 carbon atoms in one or more aromatic rings or such a group which is fused to a cycloalkyl group having from 3 to 10 carbon atoms, and the group is unsubstituted or it is substituted by at least one substituent selected from the group consisting of hydroxy groups, halogen atoms, lower alkoxy groups, lower alkylthio groups, lower alkyl groups, alkanoyloxy groups, mercapto groups, alkanoylthio groups, lower alkylsulfinyl groups, lower alkyl groups having at least one substituent selected from the group consisting of cycloalkloxy groups, lower haloalkoxy groups, and lower alkylenedioxy groups.

In some embodiments, the 1,2-diphenylpyrrole derivative is selected from the group consisting of compounds 2-1-2-213 of Table 2 as disclosed in U.S. Pat. No. 6,887,893, which is herein incorporated in its entirety by reference.

In one embodiment, the 1,2-diphenylpyrrole derivative is selected from the group consisting of: 4-methyl-2-(4-methylphenyl)-1-(4-sulfamoylphenyl)pyrrole; 2-(4-methoxyphenyl)-4-methyl-1-(4-sulfamoylphenyl)pyrrole; 2-(4-chlorophenyl)-4-methyl-1-(4-sulfamoylphenyl)pyrrole; 4-methyl-2-(4-methylthiophenyl)-1-(4-sulfamoylphenyl)pyrrole; 2-(4-ethoxyphenyl)-4-methyl-1-(4-sulfamoylphenyl)pyrrole; 2-(4-methoxy-3-methylphenyl)-4-methyl-1-(4-sulfamoylphenyl)pyrrole; 2-(3-fluoro-4-methoxyphenyl)-4-methyl-1-(4-sulfamoylphenyl)pyrrole; 2-(3,4-dimethylphenyl)-4-methyl-1-(4-sulfamoylphenyl)pyrrole; 4-methyl-1-(4-methylthiophenyl)-2-(4-sulfamoylphenyl)pyrrole; 1-(4-acetylaminosulfonylphenyl)-4-methyl-2-(4-methoxyphenyl)pyrrole; and 1-(4-acetylaminosulfonylphenyl)-4-methyl-2-(3,4-dimethylphenyl)pyrrole. In another embodiment, the invention provides a method wherein the 1,2-diphenylpyrrole derivative is 2-(4-ethoxyphenyl)-4-methyl-1-(4-sulfamoylphenyl)-pyrrole.

In another embodiment, the 1,2-diphenylpyrrole derivative is 2-(4-ethoxyphenyl)-4-methyl-1-(4-sulfamoylphenyl)-pyrrole.

The methods for synthesizing 1,2-diphenylpyrrole derivatives, illustrated herein, are described in the Examples section and in U.S. Pat. No. RE39,420, which is incorporated herein by reference in its entirety.

In one embodiment the inhibitor of both EGFR [ErbB1] and BER2 [ErbB2] has the following formula:

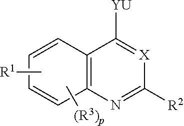

or a salt or solvate thereof;
wherein X is N or CH;
Y is a group W(CH₂), (CH₂)W, or W, in which W is O, S(O)ₘ wherein m is 0, 1 or 2, or NR^{a} wherein
R^{a} is hydrogen or a C1-8 alkyl group;
R¹ represents a 5- or 6-membered heterocyclic ring containing 1 to 4 heteroatoms selected from N, O or S(O)m, wherein m is as defined above, with the provisos that the ring does not have two adjacent O or S(O)ₘ atoms and that where the ring has only N as heteroatom(s) the ring is C-linked to the quinazoline or quinoline ring, R¹ is optionally substituted by one or more R³ groups;
each R³ is independently selected from the group consisting of amino, hydrogen, halogen, hydroxy, nitro, carboxy, formyl, cyano, trifluoromethyl, trifluoromethoxy, carbamoyl, ureido, guanidino, C1-8 alkyl, C1-8 alkoxy, C3-8 cycloalkoxyl, C4-8 alkylcycloalkoxy, C1-8 alkylcarbonyl, C1-8 alkoxycarbonyl, N—C1-4 alkylcarbamoyl, N,N-di-(C1-4 alkyl)carbamoyl, hydroxyamino, C1-4 alkoxyamino, C2-4 alkanoyloxyamino, C1-4 alkylamino, di(C1-4 alkyl)amino, di-(C1-4 alkyl)amino-C1-4 alkylene-(C1-4 alkyl)amino, C1-4 alkylamino-C1-4 alkylene-(C1-4 alkyl)amino, hydroxy-C1-4 alkylene-(C1-4 alkyl)amino, phenyl, phenoxy, 4-pyridon-1-yl, pyrrolidin-1-yl, piperidino, morpholino, thiomorpholino, thiomorpholino-1-oxide, thiomorpholino-1,1-dioxide, piperazin-1-yl, 4-C1-4 alkylpiperazin-1-yl, dioxolanyl, C1-8 alkylthio, arylthio, C1-4 alkylsulphinyl, C1-4 alkylsulphonyl, arylsulphonyl, arylsulphinyl, halogen-C1-4 alkyl, hydroxy-C1-4 alkyl, C2-4 alkanoyloxy-C1-4 alkyl, C1-4 alkoxy-C1-4 alkyl, carboxy-C1-4 alkyl, formyl-C1-4 alkyl, C1-4 alkoxycarbonyl-C1-4-alkyl, carbamoyl-C1-4 alkyl, N—C1-4 alkylcarbamoyl-C1-4 alkyl, N,N-di-(C1-4 alkyl)carbamoyl-C1-4 alkyl, amino-C1-4 alkyl, C1-4 alkylamino-C1-4 alkyl, di-(C1-4 alkyl)amino-C1-4 alkyl, phenyl-C1-4 alkyl, 4-pridon-1-yl-C1-4 alkyl, pyrrolidin-1-yl-C1-4 alkyl, imidazol-1-yl-C1-4 alkyl, piperidino-C1-4 alkyl, morpholino-C1-4 alkyl, thiomorpholino-C1-4 alkyl, thiomorpholino-1-oxide-C1-4 alkyl, thiomorpholino-1,1-dioxide-C1-4 alkyl, piperazin-1-yl-C1-4 alkyl, 4-C1-4 alkylpiperazin-1-yl-C1-4 alkyl, hydroxy-C2-4 alkoxy-C1-4 alkyl, C1-4 alkoxy-C2-4 alkoxy-C1-4 alkyl, hydroxy-C2-4 alkylamino-C1-4 alkyl, C1-4 alkoxy-C2-4 alkylamino-C1-4 alkyl, C1-4 alkylthio-C1-4 alkyl, C1-4 alkylsulphinyl-C1-4 alkyl, C1-4 alkylsulphonyl-C1-4 alkyl, hydroxy-C2-4 alkylthio-C1-4 alkyl, C1-4 alkoxy-C2-4 alkylthio-C1-4 alkyl, phenoxy-C1-4 alkyl, anilino-C1-4 alkyl, phenylthio-C1-4 alkyl, cyano-C1-4 alkyl, halogeno-C2-4 alkoxy, hydroxy-C2-4 alkoxy, C2-4 alkoxy, C2-4 alkanoyloxy-C2-4 alkoxy, C1-4 alkoxy-C2-4 alkoxy, carboxy-C1-4 alkoxy, formyl-C1-4 alkoxy, C1-4 alkoxycarbonyl-C1-4 alkoxy, carbamoyl-C1-4 alkoxy, N—C1-4 alkylcarbamoyl-C1-4 alkoxy, N,N-di-(C1-4 alkyl)carbamoyl-C1-4 alkoxy, amino-C2-4 alkoxy, C1-4 alkylamino-C2-4 alkoxy, di-(C1-4 alkyl)amino-C2-4 alkoxy, di-(C1-4 alkyl-C2-4 alkoxy)amino-C2-4 alkoxy, C2-4 alkanoyloxy, hydroxy-C2-4 alkanoyloxy, C1-4 alkoxy-C2-4 alkanoyloxy, phenyl-C1-4 alkoxy, phenoxy-C2-4 alkoxy, anilino-C2-4 alkoxy, phenylthio-C2-4 alkoxy, 4-pyridon-1-yl-C2-4 alkoxy, piperidino-C2-4 alkoxy, morpholino-C2-4 alkoxy, thiomorpholino-C2-4 alkoxy, thiomorpholino-1-oxide-C2-4 alkoxy, thiomorpholino-1,1-dioxide-C2-4 alkoxy, piperazin-1-yl-C2-4 alkoxy, 4-C1-4 alkylpiperazin-1-yl-C2-4 alkoxy, pyrrolidin-1-yl-C2-4 alkoxy, imidazol-1-yl-C2-4 alkoxy, halogeno-C2-4 alkylamino, hydroxy-C2-4 alkylamino, C2-4 alkanoyloxy-C2-4 alkylamino, C1-4 alkoxy-C2-4 alkylamino, carboxy-C1-4 alkylamino, C1-4 alkoxycarbonyl-C1-4 alkylamino, carbamoyl-C1-4 alkylamino, N—C1-4 alkylcarbamoyl-C1-4 alkylamino, N,N-di-(C1-4 alkyl)carbamoyl-C1-4 alkylamino, amino-C2-4 alkylamino, C1-4 alkylamino-C2-4 alkylamino, di-(C1-4 alkyl)amino-C2-4 alkylamino, phenyl-C1-4 alkylamino, phenoxy-C2-4 alkylamino, anilino-C2-4 alkylamino, 4-pyridon-1-yl-C2-4 alkylamino, pyrrolidin-1-yl-C2-4 alkylamino, imidazol-1-yl-C2-4 alkylamino, piperidino-C2-4 alkylamino, morpholino-C2-4 alkylamino, thiomorpholino-C2-4 alkylamino, thiomorpholino-1-oxide-C2-4 alkylamino, thiomorpholino-1,1-dioxide-C2-4 alkylamino, piperazin-1-yl-C2-4 alkylamino, 4-(C1-4 alkyl)piperazin-1-yl-C2-4 alkylamino, phenylthio-C2-4 alkylamino, C2-4 alkanoylamino, C1-4 alkoxycarbonylamino, C1-4 alkylsulphonylamino, C1-4 alkylsulphinylamino, benzamido, benzenesulphonamido, 3-phenylureido, 2-oxopyrrolidin-1-yl, 2,5-dioxopyrrolidin-1-yl, halogeno-C2-4 alkanoylamino, hydroxy-C2-4 alkanoylamino, hydroxy-C2-4 alkanoyl-(C1-4 alkyl)-amino, C1-4 alkoxy-C2-4 alkanoylamino, carboxy-C2-4 alkanoylamino, C1-4 alkoxycarbonyl-C2-4 alkanoylamino, carbamoyl-C2-4 alkanoylamino, N—C1-4 alkylcarbamoyl-C2-4 alkanoylamino, N,N-di-(C1-4 alkyl)carbamoyl-C2-4 alkanoylamino, amino-C2-4 alkanoylamino, C1-4 alkylamino-C2-4 alkanoylamino or di-(C1-4 alkyl)amino-C2-4 alkanoylamino; and
wherein said benzamido or benzenesulphonamido substitutent or any anilino, phenoxy or phenyl group on a R³ substituent may optionally have one or two halogen, C1-4 alkyl or C1-4 alkoxy substituents; and wherein any substituent having a heterocyclic ring may optionally have one or two halogen, C1-4 alkyl or C1-4 alkoxy substituents on said ring;
and wherein any substituent having a heterocyclic ring may optionally have one or two oxo or thioxo substituents on said ring;
or R³ represents a group selected from M1-M2-M3-M4, M1-M5 or M1-M2-M3′-M6 wherein
M1 represents a C1-4 alkyl group, wherein optionally a CH₂ group is replaced by a CO group;
M2 represents NR¹² or CR¹²R¹³, in which R¹² and R¹³ each independently represent H or C1-4 alkyl;
M3 represents a C1-4 alkyl group;
M3′ represents a C1-4 alkyl group or is absent;
M4 represents CN, NR¹²S(O)ₘR¹³, S(O)ₘNR¹⁴R¹⁵, CONR¹⁴R¹⁵, S(O)ₘR¹³ or CO₂R¹³, in which
R¹², R¹³ and m are as above defined and R¹⁴ and R¹⁵ each independently represent H or C1-4 alkyl, or
R¹⁴ and R¹⁵ together with the nitrogen atom to which they are attached form a 5- or 6-membered ring optionally containing 1 or 2 additional heteroatoms selected from N, O or S(O)ₘ in which ring any nitrogen atom present may optionally be substituted with a C1-4 alkyl group, and which ring may optionally contained one or two oxo or thioxo substituents;
M5 represents the group NR¹⁴R¹⁵, wherein R¹⁴ and R¹⁵ are as defined above, or M5 represents the group

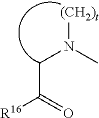

in which t represents 2 to 4 and R¹⁶ represents OH, OC1-4 alkyl or NR¹⁴R¹⁵; and
M6 represents a C3-6 cycloalkyl group, the group NR¹⁴R15, wherein R¹⁴ and R¹⁵ are as defined above, or a 5- or 6-membered heterocyclic ring system containing 1 to 4 heteroatoms selected from N, O or S;
and p is 0 to 3; or when p is 2 or 3, two adjacent R³ groups together form an optionally substituted methylenedioxy or ethylenedioxy group;
R² is selected from the group consisting of hydrogen, halogen, trifluoromethyl, C1-4 alkyl and C1-4 alkoxy;
U represents phenyl or a 5 to 10-membered mono or bicyclic ring system in which one or more of the carbon atoms is optionally replaced by a heteroatom independently selected from N, O and S(O)ₘ, wherein m is 0, 1 or 2, and wherein U is substituted by at least one independently selected R⁶ group and U is optionally substituted by at least one independently selected R⁴ group;
each R⁴ is independently hydrogen, hydroxy, halogen, C1-4 alkyl, C1-4 alkoxy, C1-4 alkylamino, di-[C1-4 alkyl]amino, C1-4 alkylthio, C1-4 alkylsulphinyl, C1-4 alkylsulphonyl, C1-4 alkylcarbonyl, C1-4 alkylcarbamoyl, di-[C1-4 alkyl]carbamoyl, carbamyl, C1-4 alkoxycarbonyl, cyano, nitro or trifluoromethyl;
each R⁶ is independently a group ZR⁷ wherein Z is joined to R⁷ through a (CH₂)ₚ group in which p is 0, 1 or 2 and Z represents a group V(CH₂), V(CF₂), (CH₂)V, (CF₂)V, V(CRR′), V(CHR) or V where R and R′ are each C1-4 alkyl and in which V is a hydrocarbyl group containing 0, 1 or 2 carbon atoms, carbonyl, dicarbonyl, CH(OH), CH(CN), sulphonamide, amide, O, S(O)ₘ or NR^{b} where R^{b} is hydrogen or R^{b} is C1-4 alkyl; and R⁷ is an optionally substituted C3-6 cycloalkyl; or an optionally substituted 5, 6, 7, 8, 9 or 10-membered carbocyclic or heterocyclic moiety;
or R⁶ is a group ZR⁷ in which Z is NR^{b}, and NR^{b} and R⁷ together form an optionally substituted 5, 6, 7, 8, 9 or 10-membered carbocyclic or heterocyclic moiety.

In another embodiment the inhibitor of both EGFR [ErbB1] and HER2 [ErbB2] as described above can be further refined wherein R¹ is a 5- or 6-membered heterocyclic ring substituted by one or more R³ groups selected from C1-4 alkylsulphinyl-C1-4 alkyl or C1-4 alkylsulphonyl-C1-4 alkyl.

In another embodiment the inhibitor of both EGFR [ErbB1] and HER2 [ErbB2] as described above can be further refined wherein X represents N; Y represents NR^{a}, wherein R^{a} is hydrogen or C1-4 alkyl; R¹ represents furan, thiazole, thiophene, pyrrole, pyridine, pyrimidine, pyrazine, imidazole, oxazole, isoxazole, oxadiazole, tetrazole, triazole, dioxolane or a partially or fully hydrogenated derivative of any of these groups, optionally substituted with an R³ group selected from methylsulphonylethylaminomethyl, methylsulphonylethylamino-carbonyl, methylsulphinylethylamino-methyl, methylsulphinylethylamino-carbonyl, methylsulphonylpropylamino-methyl, methylsulphinylpropylamino-methyl, methylsulphonylpropyamino-carbonyl, methylsulphinylpropylamino-carbonyl, methylsulphonylethyl-(methylamino)-methyl, methylsulphonylethyl-(methylamino)-carbonyl, methylsulphinylethyl-(methylamino)-methyl, methylsulphinylethyl-(methylamino)-carbonyl, methylsulphonylpropyl-(methylamino)-methyl, methylsulphinylpropyl-(methylamino)-methyl, methylsulphonylpropyl-(methylamino)-carbonyl, methylsulphinylpropyl-(methylamino)-carbonyl, methylsulphonamidoethylamino-methyl, methylsulphonamidopropylamino-methyl, aminosulphonylethylaminomethyl, methylaminosulphonylethylaminomethyl, sarcosinamidomethyl, glycinylmethyl, glycinamidomethyl, glycinylmethyl methyl ester acetylaminoethylaminomethyl, piperazinylmethyl, methylpiperazinylmethyl, piperidinylmethyl, pyridylmethyl, N-(prolinamido)methyl, (N,N-dimethyl-prolinamido)methyl, pyridylaminomethyl, cyclopropylaminomethyl, N-(piperidin-4-yl)-N-methylaminomethyl, N,N-dimethylaminoprop-2-ylaminomethyl, N-(2-dimethylaminoethyl)-N-ethylaminomethyl, isopropylacetamido, N-morpholinylacetamido or tetrahydrofuranomethylaminomethyl and optionally further substituted by one or more C1-4 alkyl groups; p is 0; R² represents hydrogen; R⁴ represents hydrogen, halo or methyl; U represents phenyl, indolyl, benzimidazolyl or indazolyl; and R⁶ represents phenyl, benzyl, α-methylbenzyl, fluorobenzyl, difluorobenzyl, pyridylmethyl, benzenesulphonyl, phenoxy, fluorophenoxy, benzyloxy or fluorobenzyloxy. In another embodiment the inhibitor of both EGFR [ErbB1] and HER2 [ErbB2] is selected from the group:

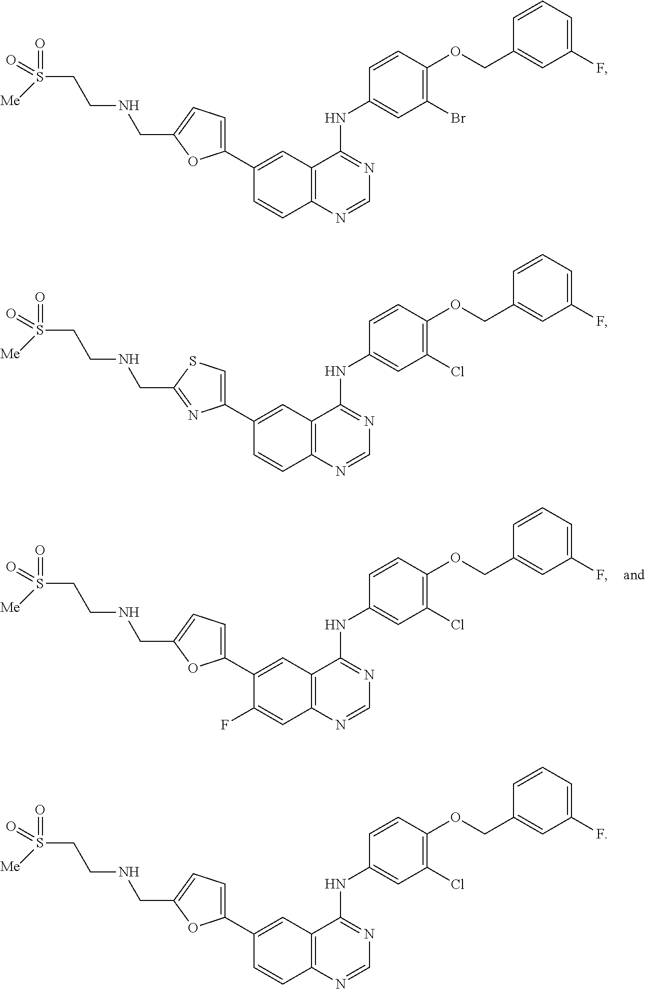

In yet another embodiment the inhibitor of both EGFR [ErbB1] and HER2 [ErbB2] is lapatinib.

### Methods of Use

The invention provides a method for treating a subject having a tumor, a tumor-related disorder, and/or cancer, comprising administering to the subject, a therapeutically effective amount of a combination comprising 2-(4-ethoxyphenyl)-4-methyl-1-(4-sulfamoylphenyl)-pyrrole and lapatinib.

In one embodiment, the invention provides a method for treating a subject having a tumor, a tumor-related disorder, and/or cancer, comprising administering to the subject, a therapeutically effective amount of a combination comprising a 1,2-diphenylpyrrole derivative and an inhibitor of both EGFR [ErbB1] and HER2 [ErbB2] wherein the 1,2-diphenylpyrrole derivative is 2-(4-ethoxyphenyl)-4-methyl-1-(4-sulfamoylphenyl)-pyrrole.

In another embodiment, the invention provides a method for treating a subject having a tumor, a tumor-related disorder, and/or cancer, comprising administering to the subject, a therapeutically effective amount of a combination comprising a 1,2-diphenylpyrrole derivative and an inhibitor of both EGFR [ErbB1] and HER2 [ErbB2] wherein the inhibitor of both EGFR [ErbB1] and HER2 [ErbB2] is lapatimib.

In yet another embodiment, the invention provides a method for treating a subject having a tumor, tumor-related disorders, and/or cancer, comprising administering to the subject, a therapeutically effective amount of a combination comprising a 1,2-diphenylpyrrole derivative and an inhibitor of both EGFR [ErbB1] and HER2 [ErbB2] or their respective pharmaceutically acceptable salt, solvate or prodrug.

In one embodiment, the invention provides a method for treating a subject having a tumor, a tumor-related disorder, and/or cancer, comprising administering to the subject, a therapeutically effective amount of a combination comprising a 1,2-diphenylpyrrole derivative and an inhibitor of both EGFR [ErbB1] and HER2 [ErbB2] wherein the 1,2-diphenylpyrrole derivative is selected from the group consisting of: 4-methyl-2-(4-methylphenyl)-1-(4-sulfamoylphenyl)pyrrole; 2-(4-methoxyphenyl)-4-methyl-1-(4-sulfamoylphenyl)pyrrole; 2-(4-chlorophenyl)-4-methyl-1-(4-sulfamoylphenyl)pyrrole; 4-methyl-2-(4-methylthiophenyl)-1-(4-sulfamoylphenyl)pyrrole; 2-(4-ethoxyphenyl)-4-methyl-1-(4-sulfamoylphenyl)pyrrole; 2-(4-methoxy-3-methylphenyl)-4-methyl-1-(4-sulfamoylphenyl)pyrrole; 2-(3-fluoro-4-methoxyphenyl)-4-methyl-1-(4-sulfamoylphenyl)pyrrole; 2-(3,4-dimethylphenyl)-4-methyl-1-(4-sulfamoylphenyl)pyrrole; 4-methyl-1-(4-methylthiophenyl)-2-(4-sulfamoylphenyl)pyrrole; 1-(4-acetylaminosulfonylphenyl)-4-methyl-2-(4-methoxyphenyl)pyrrole; and 1-(4-acetylaminosulfonylphenyl)-4-methyl-2-(3,4-dimethylphenyl)pyrrole.

In yet another embodiment, the invention provides a method for treating a subject having a tumor, a tumor-related disorder, and/or cancer, comprising administering to the subject, a therapeutically effective amount of a combination comprising a 1,2-diphenylpyrrole derivative and an inhibitor of both EGFR [ErbB1] and HER2 [ErbB2] wherein the 1,2-diphenylpyrrole derivative and the inhibitor of both EGFR [ErbB1] and HER2 [ErbB2] are administered sequentially in either order or simultaneously. In a further embodiment, the invention provides a method for treating a subject having a tumor, a tumor-related disorder, and/or cancer, comprising administering to the subject, a therapeutically effective amount of a combination comprising a 1,2-diphenylpyrrole derivative and an inhibitor of both EGFR [ErbB1] and HER2 [ErbB2] wherein the 1,2-diphenylpyrrole derivative is administered first. In one embodiment, the invention provides a method for treating a subject having a tumor, a tumor-related disorder, and/or cancer, comprising administering to the subject, a therapeutically effective amount of a combination comprising a 1,2-diphenylpyrrole derivative and an inhibitor of both EGFR [ErbB1] and HER2 [ErbB2] wherein the inhibitor of both EGFR [ErbB1] and HER2 [ErbB2] is administered first. In another embodiment, the invention provides a method for treating a subject having a tumor, a tumor-related disorder, and/or cancer, comprising administering to the subject, a therapeutically effective amount of a combination comprising a 1,2-diphenylpyrrole derivative and an inhibitor of both EGFR [ErbB1] and HER2 [ErbB2] wherein administering the combination enhances treatment of the subject in comparison to a treatment of either a 1,2-diphenylpyrrole derivative or an inhibitor of both EGFR [ErbB1] and HER2 [ErbB2] alone. In yet another embodiment, the invention provides a method for treating a subject having a tumor, a tumor-related disorder, and/or cancer, comprising administering to the subject, a therapeutically effective amount of a combination comprising a 1,2-diphenylpyrrole derivative and an inhibitor of both EGFR [ErbB1] and HER2 [ErbB2] wherein administering the combination reduces the side effects of the treatment of tumors, tumor-related disorders, and/or cancer.

In one embodiment, the invention provides a method for treating a subject having a tumor, a tumor-related disorder, and/or cancer, wherein the tumors, tumor-related disorders, and/or cancer are characterized as overexpressing HER2/neu.

In one embodiment, the invention provides a method for treating a subject having a tumor, a tumor-related disorder, and/or cancer, comprising administering to the subject, a therapeutically effective amount of a combination comprising a 1,2-diphenylpyrrole derivative and an inhibitor of both EGFR [ErbB1] and HER2 [ErbB2] wherein the cancer is selected from the group consisting of: oral cancer, prostate cancer, rectal cancer, non-small cell lung cancer, lip and oral cavity cancer, liver cancer, lung cancer, anal cancer, kidney cancer, vulvar cancer, breast cancer, oropharyngeal cancer, nasal cavity and paranasal sinus cancer, nasopharyngeal cancer, urethra cancer, small intestine cancer, bile duct cancer, bladder cancer, ovarian cancer, laryngeal cancer, hypopharyngeal cancer, gallbladder cancer, colon cancer, colorectal cancer, head and neck cancer, parathyroid cancer, penile cancer, vaginal cancer, thyroid cancer, pancreatic cancer, esophageal cancer, Hodgkin's lymphoma, leukemia-related disorders, mycosis fungoides, and myelodysplastic syndrome.

In one embodiment, the invention provides a method for treating a subject having a tumor, a tumor-related disorder, and/or cancer, comprising administering to the subject, a therapeutically effective amount of a combination comprising a 1,2-diphenylpyrrole derivative and an inhibitor of both EGFR [ErbB1] and HER2 [ErbB2] wherein the cancer is selected from breast cancer, ovarian cancer, endometrial cancer, prostate cancer, gastric cancer, salivary gland cancer, pancreatic cancer, colorectal cancer, non-small cell lung cancers, oral cancers, and cutaneous squamous cell carcinoma.

In another embodiment, the invention provides a method for treating a subject having a tumor, a tumor-related disorder, and/or cancer, comprising administering to the subject, a therapeutically effective amount of a combination comprising a 1,2-diphenylpyrrole derivative and an inhibitor of both EGFR [ErbB1] and HER2 [ErbB2] wherein the cancer is non-small cell lung cancer, pancreatic cancer, breast cancer, ovarian cancer, colorectal cancer, and head and neck cancer.

In one embodiment, the invention provides a method for treating a subject having a tumor, a tumor-related disorder, and/or cancer, comprising administering to the subject, a therapeutically effective amount of a combination comprising a 1,2-diphenylpyrrole derivative and an inhibitor of both EGFR [ErbB1] and HER2 [ErbB2] wherein the cancer is breast cancer.

In one embodiment, the invention provides a method for treating a subject having cancer wherein the cancer is a carcinoma, a tumor, a neoplasm, a lymphoma, a melanoma, a glioma, a sarcoma, and a blastoma.

In one embodiment, the invention provides a method for treating a subject having a carcinoma wherein the carcinoma is selected from the group consisting of: carcinoma, adenocarcinoma, adenoid cystic carcinoma, adenosquamous carcinoma, adrenocortical carcinoma, well differentiated carcinoma, squamous cell carcinoma, serous carcinoma, small cell carcinoma, invasive squamous cell carcinoma, large cell carcinoma, islet cell carcinoma, oat cell carcinoma, squamous carcinoma, undifferentiatied carcinoma, verrucous carcinoma, renal cell carcinoma, papillary serous adenocarcinoma, merkel cell carcinoma, hepatocellular carcinoma, soft tissue carcinomas, bronchial gland carcinomas, capillary carcinoma, bartholin gland carcinoma, basal cell carcinoma, carcinosarcoma, papilloma/carcinoma, clear cell carcinoma, endometrioid adenocarcinoma, mesothelial, metastatic carcinoma, mucoepidermoid carcinoma, cholangiocarcinoma, actinic keratoses, cystadenoma, and hepatic adenomatosis.

In one embodiment, the invention provides a method for treating a subject having a tumor wherein the tumor is selected from the group consisting of: astrocytic tumors, malignant mesothelial tumors, ovarian germ cell tumor, supratentorial primitive neuroectodermal tumors, Wilm's tumor, pituitary tumors, extragonadal germ cell tumor, gastrinoma, germ cell tumors, gestational trophoblastic tumor, brain tumors, pineal and supratentorial primitive neuroectodermal tumors, pituitary tumor, somatostatin-secreting tumor, endodermal sinus tumor, carcinoids, central cerebral astrocytoma, glucagonoma, hepatic adenoma, insulinoma, medulloepithelioma, plasmacytoma, vipoma, and pheochromocytoma.

In one embodiment, the invention provides a method for treating a subject having a neoplasm wherein the neoplasm is selected from the group consisting of: intaepithelial neoplasia, multiple myeloma/plasma cell neoplasm, plasma cell neoplasm, interepithelial squamous cell neoplasia, endometrial hyperplasia, focal nodular hyperplasia, hemangioendothelioma, and malignant thymoma.

In one embodiment, the invention provides a method for treating a subject having a lymphoma wherein the lymphome is selected from the group consisting of: nervous system lymphoma, AIDS-related lymphoma, cutaneous T-cell lymphoma, non-Hodgkin's lymphoma, lymphoma, and Waldenstrom's macroglobulinemia.

In one embodiment, the invention provides a method for treating a subject having a melanoma wherein the melaoma is selected from the group consisting of: acral lentiginous melanoma, superficial spreading melanoma, uveal melanoma, lentigo maligna melanomas, melanoma, intraocular melanoma, adenocarcinoma nodular melanoma, and hemangioma.

In one embodiment, the invention provides a method for treating a subject having a sarcoma wherein the srcoma is selected from the group consisting of: adenomas, adenosarcoma, chondosarcoma, endometrial stromal sarcoma, Ewing's sarcoma, Kaposi's sarcoma, leiomyosarcoma, rhabdomyosarcoma, sarcoma, uterine sarcoma, osteosarcoma, and pseudosarcoma.

In one embodiment, the invention provides a method for treating a subject having a glioma wherein the glioma is selected from the group consisting of: glioma, brain stem glioma, and hypothalamic and visual pathway glioma.

In one embodiment, the invention provides a method for treating a subject having a blastoma wherein the blastoma is selected from the group consisting of: pulmonary blastoma, pleuropulmonary blastoma, retinoblastoma, neuroblastoma, medulloblastoma, glioblastoma, and hemangiblastomas.

In one embodiment the inhibitor of both EGFR [ErbB1] and HER2 [ErbB2] is a small molecule compound. In another embodiment the inhibitor of both EGFR [ErbB1] and HER2 [ErbB2] is a small molecule compound selected from the group consisting of: GW2974, gefitinib, AEE788, HKI-272, BIBW-2992 and PKI-166 or their pharmaceutically acceptable salts, solvates, or prodrugs.

In one embodiment the invention provides a method of inducing differentiation of tumor cells, the method comprising contacting the cells with an effective amount of a combination comprising a 1,2-diphenylpyrrole derivative and an inhibitor of both EGFR [ErbB1] and HER2 [ErbB2] whereby the combination induces differentiation of tumor cells. In one embodiment, the invention provides a method of inducing differentiation of tumor cells, the method comprising contacting the cells with an effective amount of a combination comprising a 1,2-diphenylpyrrole derivative and an inhibitor of both EGFR [ErbB1] and HER2 [ErbB2] wherein the 1,2-diphenylpyrrole derivative is 2-(4-ethoxyphenyl)-4-methyl-1-(4-sulfamoylphenyl)-pyrrole and the inhibitor of both EGFR [ErbB1] and HER2 [ErbB2] is lapatinib.

In one embodiment the invention provides a method of inhibiting proliferation of cancer cells, the method comprising contacting a cancer cell with a combination comprising a 1,2-diphenylpyrrole derivative and an inhibitor of both EGFR [ErbB1] and HER2 [ErbB2] whereby the combination inhibits proliferation of cancer cells. In one embodiment, the invention provides a method of inhibiting proliferation of cancer cells, the method comprising contacting a cancer cell with a combination comprising a 1,2-diphenylpyrrole derivative and an inhibitor of both EGFR [ErbB1] and HER2 [ErbB2] wherein the 1,2-diphenylpyrrole derivative is 2-(4-ethoxyphenyl)-4-methyl-1-(4-sulfamoylphenyl)-pyrrole and the inhibitor of both EGFR [ErbB1] and HER2 [ErbB2] is lapatinib.

In another embodiment the invention provides a method for reducing proliferation of cancer cells, the method comprising delivering to the cells a combination comprising a 1,2-diphenylpyrrole derivative and an inhibitor of both EGFR [ErbB1] and HER2 [ErbB2], whereby the reduction of cell proliferation is greater than a reduction caused by either a 1,2-diphenylpyrrole derivative alone or an inhibitor of both EGFR [ErbB1] and HER2 [ErbB2] alone. In one embodiment, the invention provides a method for reducing proliferation of cancer cells, the method comprising delivering to the cells a combination comprising a 1,2-diphenylpyrrole derivative and an inhibitor of both EGFR [ErbB1] and HER2 [ErbB2] wherein the 1,2-diphenylpyrrole derivative is 2-(4-ethoxyphenyl)-4-methyl-1-(4-sulfamoylphenyl)-pyrrole and the inhibitor of both EGFR [ErbB1] and HER2 [ErbB2] is lapatinib.

In one embodiment the invention provides a method of modulating autophosphorylation with a molecule of ATP, the method comprising delivering to a cancer cell an effective amount of a combination comprising a 1,2-diphenylpyrrole derivative and inhibitor of both EGFR [ErbB1] and HER2 [ErbB2] wherein the combination inhibits autophosphorylation with a molecule of ATP. In one embodiment, the invention provides a method of modulating autophosphorylation with a molecule of ATP, the method comprising delivering to a cancer cell an effective amount of a combination comprising a 1,2-diphenylpyrrole derivative and an inhibitor of both EGFR [ErbB1] and HER2 [ErbB2] wherein the 1,2-diphenylpyrrole derivative is 2-(4-ethoxyphenyl)-4-methyl-1-(4-sulfamoylphenyl)-pyrrole and the inhibitor of both EGFR [ErbB1] and HER2 [ErbB2] is lapatinib.

In a further embodiment the invention provides a method of inhibiting metastases of tumor cells, the method comprising administering an effective amount of a combination comprising a 1,2-diphenylpyrrole derivative and an inhibitor of both EGFR [ErbB1] and HER2 [ErbB2] such that the combination inhibits metastatic activity of tumor cells. In one embodiment, the invention provides a method of inhibiting metastases of tumor cells, the method comprising administering an effective amount of a combination comprising a 1,2-diphenylpyrrole derivative and an inhibitor of both EGFR [ErbB1] and HER2 [ErbB2] wherein the 1,2-diphenylpyrrole derivative is 2-(4-ethoxyphenyl)-4-methyl-1-(4-sulfamoylphenyl)-pyrrole and the inhibitor of both EGFR [ErbB1] and HER2 [ErbB2] is lapatinib.

In one embodiment the invention provides a method for inducing apoptosis in cancer cells, the method comprising contacting the cancer cells with a combination comprising a 1,2-diphenylpyrrole derivative and an inhibitor of both EGFR [ErbB1] and HER2 [ErbB2] sufficient to induce apoptosis. In one embodiment, the invention provides a method for inducing apoptosis in cancer cells, the method comprising contacting the cancer cells with a combination comprising a 1,2-diphenylpyrrole derivative and an inhibitor of both EGFR [ErbB1] and HER2 [ErbB2] wherein the 1,2-diphenylpyrrole derivative is 2-(4-ethoxyphenyl)-4-methyl-1-(4-sulfamoylphenyl)-pyrrole and the inhibitor of both EGFR [ErbB1] and HER2 [ErbB2] is lapatinib.

In another embodiment the invention provides a method for sensitizing EGFR [ErbB1] inhibitor resistant cancer cells to an inhibitor of both EGFR [ErbB1] and HER2 [ErbB2], the method comprising administering a combination comprising a 1,2-diphenylpyrrole derivative and an inhibitor of both EGFR [ErbB1] and HER2 [ErbB2] wherein the combination sensitizes the cancer cells to the inhibitor of both EGFR [ErbB1] and HER2 [ErbB2]. In another embodiment the invention provides a method for sensitizing HER2 [ErbB2] inhibitor resistant cancer cells to an inhibitor of both EGFR [ErbB1] and HER2 [ErbB2], the method comprising administering a combination comprising a 1,2-diphenylpyrrole derivative and an inhibitor of both EGFR [ErbB1] and HER2 [ErbB2] wherein the combination sensitizes the cancer cells to the inhibitor of both EGFR [ErbB1] and HER2 [ErbB2]. In another embodiment the invention provides a method for sensitizing cancer cells resistant to an inhibitor of both EGFR [ErbB1] and HER2 [ErbB2] to an inhibitor of both EGFR [ErbB1] and HER2 [ErbB2], the method comprising administering a combination comprising a 1,2-diphenylpyrrole derivative and an inhibitor of both EGFR [ErbB1] and HER2 [ErbB2] wherein the combination sensitizes the cancer cells to the inhibitor of both EGFR [ErbB1] and HER2 [ErbB2]. In one embodiment, the invention provides a method for sensitizing EGFR [ErbB1] inhibitor resistant cancer cells to an inhibitor of both EGFR [ErbB1] and HER2 [ErbB2], the method comprising administering a combination comprising a 1,2-diphenylpyrrole derivative and an inhibitor of both EGFR [ErbB1] and HER2 [ErbB2] wherein the 1,2-diphenylpyrrole derivative is 2-(4-ethoxyphenyl)-4-methyl-1-(4-sulfamoylphenyl)-pyrrole and the inhibitor of both EGFR [ErbB1] and HER2 [ErbB2] is lapatinib. In one embodiment, the invention provides a method for sensitizing HER2 [ErbB2] inhibitor resistant cancer cells to an inhibitor of both EGFR [ErbB1] and HER2 [ErbB2], the method comprising administering a combination comprising a 1,2-diphenylpyrrole derivative and an inhibitor of both EGFR [ErbB1] and HER2 [ErbB2] wherein the 1,2-diphenylpyrrole derivative is 2-(4-ethoxyphenyl)-4-methyl-1-(4-sulfamoylphenyl)-pyrrole and the inhibitor of both EGFR [ErbB1] and HER2 [ErbB2] is lapatinib. In one embodiment, the invention provides a method for sensitizing cancer cells resistant to an inhibitor of both EGFR [ErbB1] and HER2 [ErbB2] to an inhibitor of both EGFR [ErbB1] and HER2 [ErbB2], the method comprising administering a combination comprising a 1,2-diphenylpyrrole derivative and an inhibitor of both EGFR [ErbB1] and HER2 [ErbB2] wherein the 1,2-diphenylpyrrole derivative is 2-(4-ethoxyphenyl)-4-methyl-1-(4-sulfamoylphenyl)-pyrrole and the inhibitor of both EGFR [ErbB1] and HER2 [ErbB2] is lapatinib.

In a further embodiment the invention provides a method of modulating prostaglandin synthesis in a cancer cell, the method comprising contacting the cell with a combination comprising a 1,2-diphenylpyrrole derivative and an inhibitor of both EGFR [ErbB1] and HER2 [ErbB2] wherein the combination inhibits prostaglandin synthesis in a cancer cell. In one embodiment, the invention provides a method of modulating prostaglandin synthesis in a cancer cell, the method comprising contacting the cell with a combination comprising a 1,2-diphenylpyrrole derivative and an inhibitor of both EGFR [ErbB1] and HER2 [ErbB2] wherein the 1,2-diphenylpyrrole derivative is 2-(4-ethoxyphenyl)-4-methyl-1-(4-sulfamoylphenyl)-pyrrole and the inhibitor of both EGFR [ErbB1] and HER2 [ErbB2] is lapatinib.

In one embodiment the invention provides a method of modulating cyclooxygenase expression in a cancer cell, the method comprising delivering to the cell a combination comprising a 1,2-diphenylpyrrole derivative and an inhibitor of both EGFR [ErbB1] and HER2 [ErbB2] wherein the combination inhibits cyclooxygenase expression in a cancer cell. In one embodiment, the invention provides a method of modulating cyclooxygenase expression in a cancer cell, the method comprising delivering to the cell a combination comprising a 1,2-diphenylpyrrole derivative and an inhibitor of both EGFR [ErbB1] and HER2 [ErbB2] wherein the 1,2-diphenylpyrrole derivative is 2-(4-ethoxyphenyl)-4-methyl-1-(4-sulfamoylphenyl)-pyrrole and the inhibitor of both EGFR [ErbB1] and HER2 [ErbB2] is lapatinib.

In one embodiment the invention provides a method of modulating angiogenesis in a cancer cell, the method comprising contacting the cell with a combination comprising a 1,2-diphenylpyrrole derivative and an inhibitor of both EGFR [ErbB1] and HER2 [ErbB2] wherein the combination inhibits angiogenesis in a cancer cell. In one embodiment the invention provides a method of modulating angiogenesis in a cancer cell, the method comprising contacting the cell with a combination comprising a 1,2-diphenylpyrrole derivative and an inhibitor of both EGFR [ErbB1] and HER2 [ErbB2] wherein the 1,2-diphenylpyrrole derivative is 2-(4-ethoxyphenyl)-4-methyl-1-(4-sulfamoylphenyl)-pyrrole and the inhibitor of both EGFR [ErbB1] and HER2 [ErbB2] is lapatinib. In another embodiment the invention provides a method of reducing the dosage in conventional treatment for neoplasia and/or neoplasia related disorders in a subject, the method comprising administering to a subject a combination of a 1,2-diphenylpyrrole derivative and an inhibitor of both EGFR [ErbB1] and HER2 [ErbB2] wherein the combination reduces the dosage in conventional treatment for neoplasia and/or neoplasia-related disorders. In one embodiment, the invention provides a method of reducing the dosage in conventional treatment for neoplasia and/or neoplasia related disorders in a subject, the method comprising administering to a subject a combination of a 1,2-diphenylpyrrole derivative and an inhibitor of both EGFR [ErbB1] and HER2 [ErbB2] wherein the 1,2-diphenylpyrrole derivative is 2-(4-ethoxyphenyl)-4-methyl-1-(4-sulfamoylphenyl)-pyrrole and the inhibitor of both EGFR [ErbB1] and HER2 [ErbB2] is lapatinib.

In one embodiment the invention provides a method of treating neoplasia and/or neoplasia related disorders, the method comprising administering a combination of a 1,2-diphenylpyrrole derivative and an inhibitor of both EGFR [ErbB1] and HER2 [ErbB2]. In one embodiment, the invention provides a method of treating neoplasia and/or neoplasia related disorders, the method comprising administering a combination of a 1,2-diphenylpyrrole derivative and an inhibitor of both EGFR [ErbB1] and HER2 [ErbB2] wherein the 1,2-diphenylpyrrole derivative is 2-(4-ethoxyphenyl)-4-methyl-1-(4-sulfamoylphenyl)-pyrrole and the inhibitor of both EGFR [ErbB1] and HER2 [ErbB2] is lapatinib.

In one embodiment the invention provides a method of modulating the immune response, the method comprising administering a combination of a 1,2-diphenylpyrrole derivative and an inhibitor of both EGFR [ErbB1] and HER2 [ErbB2]. In one embodiment, the invention provides a method of modulating the immune response, the method comprising administering a combination of a 1,2-diphenylpyrrole derivative and an inhibitor of both EGFR [ErbB1] and HER2 [ErbB2] wherein the 1,2-diphenylpyrrole derivative is 2-(4-ethoxyphenyl)-4-methyl-1-(4-sulfamoylphenyl)-pyrrole and the inhibitor of both EGFR [ErbB1] and HER2 [ErbB2] is lapatinib.

### Combination Therapy

In some embodiments, the composition comprising a combination of a 1,2-diphenylpyrrole derivative and an inhibitor of both EGFR [ErbB1] and HER2 [ErbB2] described herein, has an effect that is additive of the effects of the 1,2-diphenylpyrrole derivative alone and the effects of the inhibitor of both EGFR [ErbB1] and HER2 [ErbB2] alone. In another embodiment, the invention provides a composition comprising, a combination of a 1,2-diphenylpyrrole derivative and an inhibitor of both EGFR [ErbB1] and HER2 [ErbB2] wherein the 1,2-diphenylpyrrole derivative is 2-(4-ethoxyphenyl)-4-methyl-1-(4-sulfamoylphenyl)-pyrrole and the inhibitor of both EGFR [ErbB1] and HER2 [ErbB2] is lapatinib, wherein the combination has an effect that is additive of the effects of the 2-(4-ethoxyphenyl)-4-methyl-1-(4-sulfamoylphenyl)-pyrrole alone and the effects of lapatinib alone. Combinations based on 2-(4-ethoxyphenyl)-4-methyl-1-(4-sulfamoylphenyl)-pyrrole have shown synergistic advantages superior to the effects obtained with celecoxib.

In some other embodiments, the composition comprising a combination of a 1,2-diphenylpyrrole derivative and an inhibitor of both EGFR [ErbB1] and HER2 [ErbB2] described herein, has an effect that is greater than the additive effects of the 1,2-diphenylpyrrole derivative alone and the effects of the inhibitor of both EGFR [ErbB1] and HER2 [ErbB2] alone. In another embodiment, the invention provides a composition comprising, a combination of a 1,2-diphenylpyrrole derivative and an inhibitor of both EGFR [ErbB1] and HER2 [ErbB2] wherein the 1,2-diphenylpyrrole derivative is 2-(4-ethoxyphenyl)-4-methyl-1-(4-sulfamoylphenyl)-pyrrole and the inhibitor of both EGFR [ErbB1] and HER2 [ErbB2] is lapatinib, wherein the combination has an effect that is greater than the additive effects of the 2-(4-ethoxyphenyl)-4-methyl-1-(4-sulfamoylphenyl)-pyrrole alone and the effects of lapatinib alone.

In some embodiments, the composition comprising a combination of a 1,2-diphenylpyrrole derivative and an inhibitor of both EGFR [ErbB1] and HER2 [ErbB2] described herein, has an effect that is greater than the effects of the 1,2-diphenylpyrrole derivative alone (e.g., cyclooxygenase-2 inhibition alone). In another embodiment, the invention provides a composition comprising, a combination of a 1,2-diphenylpyrrole derivative and an inhibitor of both EGFR [ErbB1] and HER2 [ErbB2] wherein the 1,2-diphenylpyrrole derivative is 2-(4-ethoxyphenyl)-4-methyl-1-(4-sulfamoylphenyl)-pyrrole and the inhibitor of both EGFR [ErbB1] and HER2 [ErbB2] is lapatinib, wherein the combination has an effect that is greater than the effects of the 2-(4-ethoxyphenyl)-4-methyl-1-(4-sulfamoylphenyl)-pyrrole alone.

In other embodiments, the composition comprising a combination of a 1,2-diphenylpyrrole derivative and an inhibitor of both EGFR [ErbB1] and HER2 [ErbB2] described herein, has an effect that is greater than the effects of the inhibitor of both EGFR [ErbB1] and HER2 [ErbB2] alone. In another embodiment, the invention provides a composition comprising, a combination of a 1,2-diphenylpyrrole derivative and an inhibitor of both EGFR [ErbB1] and HER2 [ErbB2] wherein the 1,2-diphenylpyrrole derivative is 2-(4-ethoxyphenyl)-4-methyl-1-(4-sulfamoylphenyl)-pyrrole and the inhibitor of both EGFR [ErbB1] and HER2 [ErbB2] is lapatinib, wherein the combination has an effect that is greater than the effects of lapatinib alone.

In other embodiments, the invention provides a method for treating cancer, tumors, and tumor-related disorders comprising administering a composition comprising a combination of a 1,2-diphenylpyrrole derivative and an inhibitor of both EGFR [ErbB1] and HER2 [ErbB2] described herein, wherein the combination has an effect that is additive of the effects of the 1,2-diphenylpyrrole derivative alone and the effects of the inhibitor of both EGFR [ErbB1] and HER2 [ErbB2] alone. In further embodiments, the invention provides a method for treating cancer, tumors, and tumor-related disorders comprising administering a composition comprising a combination of a 1,2-diphenylpyrrole derivative and an inhibitor of both EGFR [ErbB1] and HER2 [ErbB2] wherein the 1,2-diphenylpyrrole derivative is 2-(4-ethoxyphenyl)-4-methyl-1-(4-sulfamoylphenyl)-pyrrole and the inhibitor of both EGFR [ErbB1] and HER2 [ErbB2] is lapatinib, wherein the combination has an effect that is additive of the effects of 2-(4-ethoxyphenyl)-4-methyl-1-(4-sulfamoylphenyl)-pyrrole alone and the effects of lapatinib alone.

In some other embodiments, the invention provides a method for treating cancer, tumors, and tumor-related disorders, comprising administering a composition comprising a combination of a 1,2-diphenylpyrrole derivative and an inhibitor of both EGFR [ErbB1] and HER2 [ErbB2] described herein, wherein the combination has an effect that is greater than the additive effects of the effects of the 1,2-diphenylpyrrole derivative alone and the effects of the inhibitor of both EGFR [ErbB1] and HER2 [ErbB2] alone. In other embodiments, the invention provides a method for treating cancer, tumors, and tumor-related disorders, comprising administering a composition comprising a combination of a 1,2-diphenylpyrrole derivative and an inhibitor of both EGFR [ErbB1] and HER2 [ErbB2] wherein the 1,2-diphenylpyrrole derivative is 2-(4-ethoxyphenyl)-4-methyl-1-(4-sulfamoylphenyl)-pyrrole and the inhibitor of both EGFR [ErbB1] and HER2 [ErbB2] is lapatinib, wherein the combination has an effect that is greater than the additive effects of 2-(4-ethoxyphenyl)-4-methyl-1-(4-sulfamoylphenyl)-pyrrole alone and the effects of lapatinib alone.

In some embodiments, the invention provides a method for treating cancer, tumors, and tumor-related disorders comprising administering a composition comprising a combination of a 1,2-diphenylpyrrole derivative and an inhibitor of both EGFR [ErbB1] and HER2 [ErbB2] described herein, wherein the combination has an effect that is greater than the effects of the 1,2-diphenylpyrrole derivative alone (e.g., cyclooxygenase-2 inhibition alone). In other embodiments, the invention provides a method for treating cancer, tumors, and tumor-related disorders comprising administering a composition comprising a combination of a 1,2-diphenylpyrrole derivative and an inhibitor of both EGFR [ErbB1] and HER2 [ErbB2] wherein the 1,2-diphenylpyrrole derivative is 2-(4-etboxyphenyl)-4-methyl-1-(4-sulfamoylphenyl)-pyrrole and the inhibitor of both EGFR [ErbB1] and HER2 [ErbB2] is lapatinib, wherein the combination has an effect that is greater than the effects of is 2-(4-ethoxyphenyl)-4-methyl-1-(4-sulfamoylphenyl)-pyrrole alone.

In further embodiments, the invention provides a method for treating cancer, tumors, and tumor-related disorders comprising administering a composition comprising a combination of a 1,2-diphenylpyrrole derivative and an inhibitor of both EGFR [ErbB1] and HER2 [ErbB2] described herein, wherein the combination has an effect that is greater than the effects of the inhibitor of both EGFR [ErbB1] and HER2 [ErbB2] alone. In other embodiments, the invention provides a method for treating cancer, tumors, and tumor-related disorders comprising administering a composition comprising a combination of a 1,2-diphenylpyrrole derivative and an inhibitor of both EGFR [ErbB1] and HER2 [ErbB2] wherein the 1,2-diphenylpyrrole derivative is 2-(4-ethoxyphenyl)-4-methyl-1-(4-sulfamoylphenyl)-pyrrole and the inhibitor of both EGFR [ErbB1] and HER2 [ErbB2] is lapatinib, wherein the combination has an effect that is greater than the effects of lapatinib alone. 100661 Synergism of the composition comprising a combination of a 1,2-diphenylpyrrole derivative and an inhibitor of both EGFR [ErbB1] and HER2 [ErbB2], may be used to obtain the desired effect at doses to which side effects are minimal. For example, a patient may be treated for a disease, disorder, or condition which benefits from EGFR [ErbB1] or HER2 [ErbB2] inhibition, such as tumors, tumor-related diseases, cancer, neoplasia, while concomitantly being treated for a side effect of the EGFR [ErbB1] or HER2 [ErbB2] inhibition, such as inflammation, through the benefit of the 1,2-diphenylpyrrole derivative inhibitor. In one embodiment, the invention provides a combination of 2-(4-ethoxyphenyl)-4-methyl-1-(4-sulfamoylphenyl)-pyrrole and lapatinib which may be used to obtain the desired effect at doses to which side effects are minimal.

The composition comprising a combination of a 1,2-diphenylpyrrole derivative and an inhibitor of both EGFR [ErbB1] and HER2 [ErbB2], may be applied as a sole therapy or may involve one or more other materials and treatment agents such as but not limited to a combination of capecitabine.

Thus, the composition comprising a combination of a 1,2-diphenylpyrrole derivative and an EGFR [ErbB1] inhibitor, may be applied with one or more other anti-tumor substances, for example, those selected from, mitotic inhibitors, for example vinblastine; alkylating agents, for example, cis-platin, carboplatin, and cyclophosphamide; anti-metabolites, for example capecitabine, 5-fluorouracil, cytosine arabinoside and hydroxyurea, or, for example, anti-metabolites such as N-(5-[N-(3,4-dihydro-2-methyl-4-oxoquinazolin-6-ylmethyl)-N-methylamino]-2-thenoyl)-L-glutamic acid; growth factor inhibitors; cell cycle inhibitors; intercalating antibiotics, for example adriamycin and bleomycin; enzymes, for example interferon; aromatase inhibitors, for example letrozole, anastrozole or exemestane; monoclonal antibodies, for example trastuzumab, pertuzurnab and trastuzumab-DM1; and anti-hormones, for example anti-estrogens such as Nolvadex® (tamoxifen) or, for example anti-androgens such as Casodex® (4′-cyano-3-(4-fluorophenyl sulphonyl)-2-hydroxy-2-methyl-3′-(trifluoromethyl)propionanilide). In one embodiment, the invention provides a combination of a 1,2-diphenylpyrrole derivative and an inhibitor of both EGFR [ErbB1] and HER2 [ErbB2] wherein the 1,2-diphenylpyrrole derivative is 2-(4-ethoxyphenyl)-4-methyl-1-(4-sulfamoylphenyl)-pyrrole and the inhibitor of both EGFR [ErbB1] and HER2 [ErbB2] is lapatinib, may be applied with one or more other anti-tumor substances, for example, those selected from, mitotic inhibitors, for example vinblastine; alkylating agents, for example, cis-platin, carboplatin, and cyclophosphamide; anti-metabolites, for example capecitabine, 5-fluorouracil, cytosine arabinoside and hydroxyurea, or, for example, anti-metabolites such as N-(5-[N-(3,4-dihydro-2-methyl-4-oxoquinazolin-6-ylmethyl)-N-methylamino]-2-thenyl)-L-glutamic acid; growth factor inhibitors; cell cycle inhibitors; intercalating antibiotics, for example adriamycin and bleomycin; enzymes, for example interferon; aromatase inhibitors, for example letrozole, anastrozole or exemestane; monoclonal antibodies, for example trastuzumab, pertuzumab and trastuzumab-DM1; and anti-hormones, for example anti-estrogens such as Nolvadex® (tamoxifen) or, for example anti-androgens such as Casodex® (4′-cyano-3-(4-fluorophenyl sulphonyl)-2-hydroxy-2-methyl-3′-(trifluoromethyl)propionanilide).

In one embodiment, the invention provides a method for inhibiting abnormal cell growth in a subject comprising administering to the subject an effective amount of a composition comprising a combination of a 1,2-diphenylpyrrole derivative and an inhibitor of both EGFR [ErbB1] and HER2 [ErbB2], or their pharmaceutically acceptable salt, solvate or prodrug thereof, in combination with radiation therapy effective in inhibiting abnormal cell growth in the subject. Techniques for administering radiation therapy are known to a person of skill in the art and these techniques can be used in the combination therapy described herein.

In one embodiment the invention provides a method for treating a subject having an EGFR [ErbB1] inhibitor resistant cancer cell comprising administering to the subject a therapeutically effective amount of a composition comprising a 1,2-diphenylpyrrole derivative in combination with an inhibitor of both EGFR [ErbB1] and HER2 [ErbB2]. In one embodiment, the 1,2-diphenylpyrrole derivative is 2-(4-ethoxyphenyl)-4-methyl-1-(4-sulfamoylphenyl)-pyrrole. In another embodiment the 1,2-diphenylpyrrole derivative is 2-(4-ethoxyphenyl)-4-methyl-1-(4-sulfamoylphenyl)-pyrrole and the inhibitor of both EGFR [ErbB1] and HER2 [ErbB2] is lapatinib. In one embodiment the invention provides a method for treating a subject having a HER2 [ErbB2] inhibitor resistant cancer cell comprising administering to the subject a therapeutically effective amount of a composition comprising a 1,2-diphenylpyrrole derivative in combination with an inhibitor of both EGFR [ErbB1] and HER2 [ErbB2]. In one embodiment, the 1,2-diphenylpyrrole derivative is 2-(4-ethoxyphenyl)-4-methyl-1-(4-sulfamoylphenyl)-pyrrole. In another embodiment the 1,2-diphenylpyrrole derivative is 2-(4-ethoxyphenyl)-4-methyl-1-(4-sulfamoylphenyl)-pyrrole and the inhibitor of both EGFR [ErbB1] and HER2 [ErbB2] is lapatinib. In one embodiment the invention provides a method for treating a subject having a cancer cell resistant to an inhibitor of both EGFR [ErbB1] and HER2 [ErbB2] comprising administering to the subject a therapeutically effective amount of a composition comprising a 1,2-diphenylpyrrole derivative in combination with an inhibitor of both EGFR [ErbB1] and HER2 [ErbB2]. In one embodiment, the 1,2-diphenylpyrrole derivative is 2-(4-ethoxyphenyl)-4-methyl-1-(4-sulfamoylphenyl)-pyrrole. In another embodiment the 1,2-diphenylpyrrole derivative is 2-(4-ethoxyphenyl)-4-methyl-1-(4-sulfamoylphenyl)-pyrrole and the inhibitor of both EGFR [ErbB1] and HER2 [ErbB2] is lapatinib.

In one embodiment, the invention provides a method of the treatment of NSCLC which utilizes a therapeutically effective amount of a composition comprising a combination of 2-(4-ethoxyphenyl)-4-methyl-1-(4-sulfamoylphenyl)-pyrrole and lapatinib. Further, therapy for the treatment of NSCLC utilizes a therapeutically effective amount of a composition comprising a combination of 2-(4-ethoxyphenyl)-4-methyl-1-(4-sulfamoylphenyl)-pyrrole with lapatinib, and one of the following antineoplastic agents: bevacizumab, docetaxel, gefitinib, gemeitabine, cisplatin, carboplatin, etoposide, paclitaxel, pemetrexate, vinorelbine, or radiation therapy.

One embodiment of the invention provides a combination therapy for the treatment of colorectal cancer including surgery, followed by a regimen of a composition comprising a combination of 2-(4-ethoxyphenyl)-4-methyl-1-(4-sulfamoylphenyl)-pyrrole and lapatinib. Further, another embodiment of the invention provides a combination therapy for the treatment of colorectal cancer including surgery, followed by a regimen of a composition comprising a combination of 2-(4-ethoxyphenyl)-4-methyl-1-(4-sulfamoylphenyl)-pyrrole and lapatinib, and one or more antiangiogenic agents including an MMP inhibitor, or an integrin antagonist, cycled over a one year time period. A further embodiment of the invention provides a combination therapy for the treatment of colorectal cancer including a regimen of a composition comprising a combination of 2-(4-ethoxyphenyl)-4-methyl-1-(4-sulfamoylphenyl)-pyrrole and lapatinib, followed by surgical removal of the tumor from the colon or rectum and then followed by a regimen of a composition comprising a combination of 2-(4-ethoxyphenyl)-4-methyl-1-(4-sulfamoylphenyl)-pyrrole and lapatinib cycled over a one year time period. Another therapy for the treatment of colon cancer comprises administering a combination of therapeutically effective amounts of a composition comprising a combination of 2-(4-ethoxyphenyl)-4-methyl-1-(4-sulfamoylphenyl)-pyrrole and lapatinib.

Provided herein is a pharmaceutical composition for treating cancer comprising a combination of a 1,2-diphenylpyrrole derivative and an inhibitor of both EGFR [ErbB1] and HER2 [ErbB2] and a pharmaceutically acceptable excipient or carrier.

In one embodiment, the invention provides a pharmaceutical composition comprising a combination of 2-(4-ethoxyphenyl)-4-methyl-1-(4-sulfamoylphenyl)-pyrrole and lapatinib as an active ingredient, or a pharmaceutically acceptable salt, solvate, or prodrug thereof, in a pharmaceutically acceptable vehicle, carrier, diluent, or excipient, or a mixture thereof; and one or more pharmaceutically acceptable excipients or carriers.

In another embodiment, the 1,2-diphenylpyrrole derivative is 2-(4-ethoxyphenyl)-4-methyl-1-(4-sulfamoylphenyl)-pyrrole and a pharmaceutically acceptable excipient or carrier. In another embodiment, the inhibitor of both EFRG and HER2 [ErbB2] is lapatinib and a pharmaceutically acceptable excipient or carrier. In a further embodiment the 1,2-diphenylpyrrole derivative is 2-(4-ethoxyphenyl)-4-methyl-1-(4-sulfamoylphenyl)-pyrrole and the inhibitor of both EFRG and HER2 [ErbB2] is lapatinib and a pharmaceutically acceptable excipient or carrier.

In one embodiment, the invention provides a pharmaceutical composition for treating cancer comprising a combination of a 1,2-diphenylpyrrole derivative selected from the group consisting of: 4-methyl-2-(4-methylphenyl)-1-(4-sulfamoylphenyl)pyrrole; 2-(4-methoxyphenyl)-4-methyl-1-(4-sulfamoylphenyl)pyrrole; 2-(4-chlorophenyl)-4-methyl-1-(4-sulfamoylphenyl)pyrrole; 4-methyl-2-(4-methylthiophenyl)-1-(4-sulfamoylphenyl)pyrrole; 2-(4-ethoxyphenyl)-4-methyl-1-(4-sulfamoylphenyl)pyrrole; 2-(4-methoxy-3-methylphenyl)-4-methyl-1-(4-sulfamoylphenyl)pyrrole; 2-(3-fluoro-4-methoxyphenyl)-4-methyl-1-(4-sulfamoylphenyl)pyrrole; 2-(3,4-dimethylphenyl)-4-methyl-1-(4-sulfamoylphenyl)pyrrole; 4-methyl-1-(4-methylthiophenyl)-2-(4-sulfamoylphenyl)pyrrole; 1-(4-acetylaminosulfonylphenyl)-4-methyl-2-(4-methoxyphenyl)pyrrole; and 1-(4-acetylaminosulfonylphenyl)-4-methyl-2-(3,4-dimethylphenyl)pyrrole. In another embodiment, the invention provides a method wherein the 1,2-diphenylpyrrole derivative is 2-(4-ethoxyphenyl)-4-methyl-1-(4-sulfamoylphenyl)-pyrrole and an inhibitor of both EFRG and HER2 [ErbB2] and a pharmaceutically acceptable excipient or carrier.

### INCORPORATION BY REFERENCE

All publications, patents, and patent applications described in this specification are herein incorporated by reference to the same extent as if each individual publication, patent, or patent application was specifically and individually indicated to be incorporated by reference.

### BRIEF DESCRIPTION OF THE DRAWINGS

FIG. 1 provides graphs illustrating COX-2 expression levels in colorectal cancer. The overall 10-year survival curves of patients with Cox-2 negative and Cox-2 positive are shown for the entire cohort, P=0.0006 (A), as well as for patients with stage I/II, P=0.0271 (B), or stage III, P=0.0081 (C) disease.

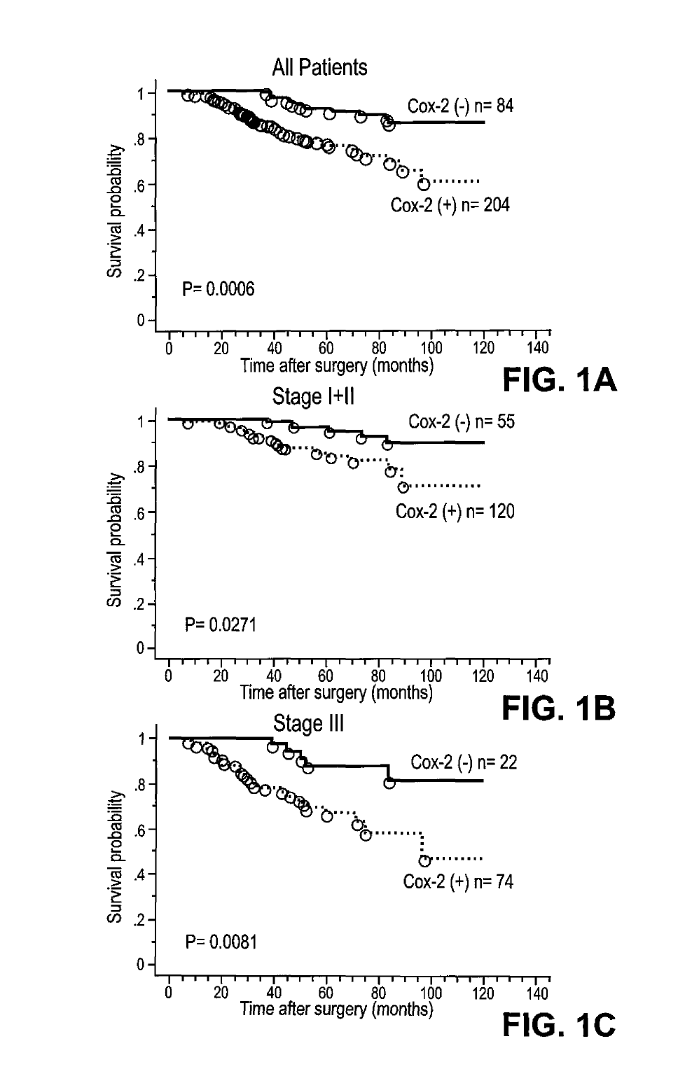

### DETAILED DESCRIPTION

Provided herein are methods of treating cancer based on the administration of a combination of a 1,2-diphenylpytrole derivative (a COX-2 selective inhibitor) and an inhibitor of both EGFR [ErbB1] and HER2 [ErbB2]. The methods may further include treatments wherein the combination is supplemented with one or more therapeutic agents or therapies. In one method, capecitabine is administered in combination with the 1,2-diphenylpyrrole and the inhibitor of both EGFR [ErbB1] and HER2 [ErbB2]. The latter combination is useful in the treatment of breast cancers associated with overexpression of HER2 [ErbB2]. The 1,2-diphenylpyrrole derivative and the inhibitor of both EGFR [ErbB1] and HER2 [ErbB2] may be provided in separate dosage forms or combined in one dosage form (e.g. a fixed dose).

To facilitate understanding of the disclosure set forth herein, a number of terms are defined below.

As used herein, “abnormal cell growth,” refers to cell growth that is independent of normal regulatory mechanisms (e.g., loss of contact inhibition), including the abnormal growth of normal cells and the growth of abnormal cells.

“Neoplasia” as described herein, is an abnormal, unregulated and disorganized proliferation of cells that is distinguished from normal cells by autonomous growth and somatic mutations. As neoplastic cells grow and divide they pass on their genetic mutations and proliferative characteristics to progeny cells. A neoplasm, or tumor, is an accumulation of neoplastic cells. In some embodiments, the neoplasm can be benign or malignant.

“Metastasis,” as used herein, refers to the dissemination of tumor cells via lymphatics or blood vessels. Metastasis also refers to the migration of tumor cells by direct extension through serous cavities, or subarachnoid or other spaces. Through the process of metastasis, tumor cell migration to other areas of the body establishes neoplasms in areas away from the site of initial appearance.

As discussed herein, “angiogenesis” is prominent in tumor formation and metastasis. Angiogenic factors have been found associated with several solid tumors such as rhabdomyosarcomas, retinoblastoma, Ewing sarcoma, neuroblastoma, and osteosarcoma. A tumor cannot expand without a blood supply to provide nutrients and remove cellular wastes. Tumors in which angiogenesis is important include solid tumors such as renal cell carcinoma, hepatocellular carcinoma, and benign tumors such as acoustic neuroma, and neurofibroma. Angiogenesis has been associated with blood-born tumors such as leukemias. It is believed that angiogenesis plays a role in the abnormalities in the bone marrow that give rise to leukemia. Prevention of angiogenesis could halt the growth of cancerous tumors and the resultant damage to the subject due to the presence of the tumor.

The term “subject” refers to an animal, including, but not limited to, a primate (e.g., human), cow, sheep, goat, horse, dog, cat, rabbit, rat, or mouse. The terms “subject” and “patient” are used interchangeably herein in reference, for example, to a mammalian subject, such as a human subject.

The terms “treat,” “treating,” and “treatment” are meant to include alleviating or abrogating a disorder, disease, or condition; or one or more of the symptoms associated with the disorder, disease, or condition; or alleviating or eradicating the cause(s) of the disorder, disease, or condition itself.

The term “therapeutically effective amount” refers to the amount of a compound that, when administered, is sufficient to prevent development of, or alleviate to some extent, one or more of the symptoms of the disorder, disease, or condition being treated. The term “therapeutically effective amount” also refers to the amount of a compound that is sufficient to elicit the biological or medical response of a cell, tissue, system, animal, or human that is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. Each component must be “pharmaceutically acceptable” in the sense of being compatible with the other ingredients of a pharmaceutical formulation. It must also be suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004).

The term “pharmaceutical composition” refers to a mixture of a compound disclosed herein with other chemical components, such as diluents or carriers. The pharmaceutical composition facilitates administration of the compound to an organism. Multiple techniques of administering a compound exist in the art including, but not limited to, oral, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

### Cyclooxygenase

Cyclooxygenase (COX) is an enzyme that is responsible for the formation of important biological mediators called prostanoids, including prostaglandins, prostacyclin and thromboxane. COX converts arachidonic acid, an ω-6 essential fatty acid, to prostaglandin H₂ (PGH₂), the precursor of the series-2 prostanoids. The enzyme contains two active sites: a heme with peroxidase activity, responsible for the reduction of PGG₂ to PGH₂, and a cyclooxygenase site, where arachidonic acid is converted into the hydroperoxy endoperoxide prostaglandin G₂ (PGG₂). The reaction proceeds through a hydrogen atom abstraction from arachidonic acid by a tyrosine radical generated by the peroxidase active site, then two oxygen molecules react with the arachidonic acid radical, giving PGG₂.

COX-1 is a constitutive enzyme responsible for biosynthesis of prostaglandins in the gastric mucosa and in the kidney among other sites. COX-2 is an enzyme that is produced by an inducible gene that is responsible for biosynthesis of prostaglandins in inflammatory cells. Inflammation causes induction of COX-2, leading to release of prostanoids (prostaglandin E2), which sensitize peripheral nociceptor terminals and produce localized pain hypersensitivity, inflammation and edema.

### Overexpression of COX-2 and Cancer

The overexpression of COX-2 and also the upstream and downstream enzymes of the prostaglandin synthesis pathway has been demonstrated in multiple cancer types and some pre-neoplastic lesions. Direct interactions of prostaglandins with their receptors through autocrine or paracrine pathways to enhance cellular survival or stimulate angiogenesis have been proposed as molecular mechanisms underlying the pro-carcinogenic functions of COX enzymes.

Studies indicate that prostaglandins synthesized by cyclooxygenase play a role in the initiation and promotion of cancer. Aberrant COX-2 expression was reported in colorectal carcinomas and adenomas, and has been detected in various human cancers, including those of the breast. Moreover, COX-2 is overexpressed in neoplastic lesions of the colon, breast, lung, prostate, esophagus, pancreas, intestine, cervix, ovaries, urinary bladder and head and neck (see Table 1 below).

TABLE 1
COX-2 Expression in Tumors

| Tumor Type | % Tissue expressing COX-2 |
|---|---|
| Colorectal Cancer | 70-95 |
| Non-small Cell Lung Cancer | 70-90 |
| Gastric Cancer | 45-75 |
| Pancreatic Cancer | 40-80 |
| Glioblastoma Multiforme | 40-70 |
| Bladder Cancer | 50-60 |
| Esophageal Cancer | 50-60 |
| Breast Cancer | 40-50 |
| Ovarian Cancer | 40-60 |
| Prostate Cancer | 40-60 |

COX-2 overexpression in murine mammary glands is sufficient to cause tumor formation. In several in vitro and animal models, COX-2 inhibitors have inhibited tumor growth and metastasis.

In addition to cancers per se, COX-2 is also expressed in the angiogenic vasculature within and adjacent to hyperplastic and neoplastic lesions indicating that COX-2 plays a role in angiogenesis. In both the mouse and rat, COX-2 inhibitors markedly inhibited bFGF-induced neovascularization. The utility of COX-2 inhibitors as chemopreventive, antiangiogenic and chemotherapeutic agents is described in the literature (Koki et al., Exp. Opin., Invest. Drugs, 1999, 8(10) 1623-38).

Additionally, several studies have suggested that COX-2 expression is associated with parameters of aggressive breast cancer, including large tumor size, positive axillary lymph node metastases, and HER2-positive tumor status. Studies of mammary tumors in mice and rats have indicated that moderate to high COX-2 expression is related to the genesis of mammary tumors that are sensitive to treatment with nonspecific and specific COX-2 inhibitors. Studies of the relationship between the HER2 [ErbB2] tyrosine kinase receptor and COX-2 have shown a link between HER2 [ErbB2] signaling and COX-2 expression in HER-2/neu-positive breast cancer (Subbaramaiah et al., J. Biol. Chem., 2002, 277, 18649-657).

### Receptor Tyrosine Kinases

Protein tyrosine kinases form a class of enzymes that catalyze the transfer of a phosphate group from ATP or GTP to the tyrosine residue located on protein substrates. Protein tyrosine kinases clearly play a role in normal cell growth. Many of the growth factor receptor proteins function as tyrosine kinases and it is by this process that they effect signaling. The interaction of growth factors with these receptors is a necessary event in normal regulation of cell growth. Under certain conditions, however, as a result of either mutation or overexpression, these receptors can become deregulated; the result of which is uncontrolled cell proliferation which can lead to tumor growth and ultimately to cancer (Wilks, Adv. Cancer Res., 1993, 60, 43). Among the growth factor receptor kinases and their proto-oncogenes that have been identified and which are targets of the combinations presented herein are the epidermal growth factor receptor kinase (EGFR [ErbB1] kinase, the protein product of the erbB oncogene), and the product produced by the erbB-2 (also referred to as the neu or HER-2) oncogene. Since the phosphorylation event is a necessary signal for cell division to occur and since overexpressed or mutated kinases have been associated with cancer, an inhibitor of this event, a protein tyrosine inhibitor, will have therapeutic value for the treatment of cancer and other diseases characterized by uncontrolled or abnormal cell growth. For example, overexpression of the receptor kinase product of the erbB-2 oncogene has been associated with human breast and ovarian cancers (Slamon et. al., Science, 1989, 244, 707). Deregulation of EGFR [ErbB1] kinase has been associated with epidermoid tumors and tumors involving other major organs. Because of the importance of the role played by deregulated receptor kinases in the pathogenesis of cancer, many recent studies have dealt with the development of specific PTK inhibitors as potential anti-cancer therapeutic agents.

Receptor tyrosine kinases span the cell membrane and possess an extracellular binding domain for growth factors such as epidermal growth factor (EGF), a transmembrane domain, and an intracellular portion which functions as a kinase to phosphorylate specific tyrosine kinase residues in proteins and hence to influence cell proliferation. The EGF receptor tyrosine kinase family has four members: EGFR [ErbB1] (HER1, erbB1); HER2 (c-erbB2, erbB2, neu); HER3 (erbB3); and HER4 (erbB4). The ErbB receptors generally transduce signals through two pathways. It is known that such kinases are frequently and aberrantly expressed in common human cancers such as breast cancer, gastrointestinal cancer of colon, rectum or stomach, leukemia, and ovarian, bronchial or pancreatic cancer. As discussed previously, epidermal growth factor receptor (EGFR [ErbB1]), is mutated and/or overexpressed in many human cancers such as brain, lung, squamous cell, bladder, gastric, breast, head and neck, oesophageal, gynecological and thyroid tumors.

### EGFR [ErbB1] and COX Pathways

The relationship between the EGFR [ErbB1] and COX pathways, to date, has not yet been fully elucidated. It has been submitted, however, that induction of COX-2 results in the production of increased levels of prostaglandins which then stimulate angiogenesis, cell proliferation and cell differentiation in an autocrine and/or paracrine manner. As described above, NSAIDs inhibit this process. Prostaglandins promote angiogenesis, along with cellular differention and proliferation. They also activate signaling through the EGFR [ErbB1]. EGFR [ErbB1] activation leads to phosphorylation on tyrosine residues by the receptor's tyrosine kinase domain, initiating a signaling pathway that includes the molecules Grb-2, SOS, the small G protein Raf. Raf activates Mitogen-Activated Protein Kinase Kinase (MAPKK) and group of nuclear transcription factors (c-myc, c-fos, c-jun); These factors initiate transcription of genes involved in the regulation of cell proliferation and differentiation. Additionally, these factors induce transcription of the COX-2 gene. These effects may significantly amplify the original EGFR [ErbB1] mediated signal and lead to pro-neoplastic effects. Inhibiting both signaling pathways could lead to a significant anti-neoplastic effect.

### HER2/neu

As indicated above, HER2/neu (also known as ErbB-2) is a member of the epidermal growth factor receptor (ErbB) family and is notable for its role in the pathogenesis of breast cancer and as a target of treatment. It is a cell membrane surface-bound receptor tyrosine kinase and is normally involved in the signal transduction pathways leading to cell growth and differentiation. HER2 is thought to be an orphan receptor, with none of the EGF family of ligands able to activate it. However, ErbB receptors dimerise on ligand binding, and HER2 is the preferential dimerisation partner of other members of the ErbB family.

The HER2 gene is a proto-oncogene located at the long arm of human chromosome 17(17q11.2-q12). Approximately 25-30 percent of breast cancers have an amplification of the HER2/neu gene or overexpression of its protein product. Overexpression of this receptor in breast cancer is associated with increased disease recurrence and worse prognosis. The oncogene neu is so-named because it was derived from a neuroglioblastoma cell line in rat. HER2 is named because it has similar structure to human epidermal growth factor receptor, or HER1. ErbB2 was named for its similarity to ErbB (avian erythroblastosis oncogene B), the oncogene later found to code for EGFR [ErbB1]. Gene cloning showed that neu, HER2, and ErbB2 were the same.

In general, there are two methods of determining HER2 status. First, measurement of gene amplification: FISH or fluorescence in-situ hybridization is a gene-based diagnostic test used to identify amplified HER2 genes and therefore excess HER2 protein. If the test shows an excess number of genes, the test is considered HER2 positive. If the test shows a normal number of genes, the test is considered HER2 negative. Second, measurement of protein expression: IHC or immunohistochemistry is a protein-based diagnostic test used to identify overexpressed HER2 protein caused by too many copies of the HER2 gene. IHC measures HER2 protein overexpression on different levels: 0, 1+, 2+ and 3+. If the test is 2+, it has been recommended that a FISH test should be conducted to confirm HER2 positive or negative status. If the tumor is 3+, it is HER2 positive.

There is increasing evidence that cyclooxygenase-2 (COX-2) may mediate the effects of HER2/neu. As discussed above, COX-2 catalyzes the conversion of arachidonic acid to prostaglandins (PGs). High levels of COX-2 and its main product, PGE2, have been found in human breast cancer cells and tumors that overexpress HER2/neu but not in normal breast tissue. It has been suggested that COX-2 overexpression increases resistance to apoptosis, particularly NO-mediated apoptosis.

HER2 has become an important target in the search for new anti-cancer therapies. Small molecule kinase inhibitors acting against HER2 alone, such as CP-654577, or against both HER2 and EGFR [ErbB1], such as lapatinib, gefitinib, AEE788, HKI-272, PKI-166 or BMW-2992, have been reported.

### HER2/neu Positive Breast Cancer

Cancers associated with overexpression of HER2/neu include breast, ovarian, endometrial, prostate, gastric, salivary gland, pancreatic, colorectal, oral and non-small cell lung cancers. Breat cancer has been a focus of anti-HER2/neu treatments.

Today, among women in the United States, breast cancer remains the most frequent diagnosed cancer. One in 8 women in the United States is at risk of developing breast cancer. Age, family history, diet, and genetic factors have been identified as risk factors for breast cancer. Breast cancer is the second leading cause of death among women. Approximately 25-30 percent of breast cancers have an amplification of the HER2/neu gene or overexpression of its protein product. Overexpression of this receptor in breast cancer is associated with increased disease recurrence and worse prognosis.

Studies of the relationship between the HER2 tyrosine kinase receptor and COX-2 have shown a link between HER2 signaling and COX-2 expression in HER2-positive breast cancer (Subbaramaiah et al., J. Biol. Chem., 2002, 277, 18649-657). This study looked at the mechanism of COX-2 regulation and found that the inductive effects of HER2 were mediated, in part, by enhanced binding of AP-1 to the cyclic AMP-response element of the COX-2 promoter. In the treatment of HER2/neu positive breast cancer, the therapies and compositions described herein may be combined with other antiangiogenic agents, or in combination with surgery, radiation therapy or with chemotherapeutic agents, including, for example, capecitabine, trastuzumab, CL-387785, paclitaxel, docetaxel, cisplatin, carboplatin, etc.

Hormone Positive Cancer

Many breast cancers require the hormone estrogen to grow. In women who have had their menopause, the main source of estrogen is through the conversion of androgens into estrogens. This process is carried out by the aromatase enzyme. In the treatment of hormone positive breast cancer, the therapies and compositions described herein may be combined with aromatase inhibitors, for example, exemestane, letrozole, and anastrozole.

Triple Negative Breast Cancer

In the treatment of triple negative breast cancer wherein the cancer is estrogen receptor-negative, progesterone receptor-negative and HER2-negative, compositions and therapies described herein may be combined with other therapeutic agents. Such agents include, by way of example only, cetuximab, paclitaxel, docetaxel, taxane formulations, for example, Abraxane® (ABI-007), Paclitaxel-Cremophor EL, Paclitaxel poliglumex, and Paclitaxel injectable emulsion (PIE). These combinations may be advantageous when the cancer association with HER2 overexpression is present but undetected due to technical limitations in the tests described above.

EGFR [ErbB1]/HER 2 neu Resistance

Clinical efficacy of ErbB1/ErbB2 kinase inhibitors is limited by the development of acquired autoresistance. Gene expression and protein analysis indicates that acquired resistance to lapatinib is mediated by increased estrogen receptor (ER) signaling rather than loss of ErbB2 expression or insensitivity of the ErbB2-MAPK-PI3K pathways to lapatinib. It has been suggested that increased ER signaling occurs in patients with ErbB2-overexpressing/ER+ breast cancers treated with lapatinib monotherapy.

As discussed above, the present disclosure provides methods of treating cancer based on the administration of a combination of a 1,2-diphenylpyrrole derivative and an inhibitor of both EGFR [ErbB1] and HER2 [ErbB2]. The disclosure provides methods wherein the the 1,2-diphenylpyrrole derivative is 2-(4-ethoxyphenyl)-4-methyl-1-(4-sulfamoylphenyl)-pyrrole and the inhibitor of both EGFR [ErbB1] and HER2 [ErbB2] is lapatinib. In certain embodiments, the disclosure provides a treatment for cancer based on administration of the compound 2-(4-ethoxyphenyl)-4-methyl-1-(4-sulfamoylphenyl)-pyrrole and lapatinib further in combination with capecitabine. The latter combination is useful in the treatment of breast cancers associated with overexpression of HER2 [ErbB2]. The 1,2-diphenylpyrrole derivative and the inhibitor of both EGFR [ErbB1] and HER2 [ErbB2] may be provided in separate dosage forms or combined in one dosage form (e.g.; a fixed dose). Additionally, in the treatment of resistant breast cancer a composition comprising a combination of 2-(4-ethoxyphenyl)-4-methyl-1-(4-sulfamoylphenyl)-pyrrole and lapatinib can be used to treat the disease in combination with aromatase inhibitors, for e.g., exemestane, letrozole, or anastrozole. Other therapies that may be advantageously combined with the present are described below.

Additional Therapy

Available treatments for breast cancer that may be advantageously employed in combination with the therapies and compositions disclosed herein include, without limitation, radiation therapy, chemotherapy, hormone therapy, antibody therapy, and tyrosine kinase inhibitors as adjuvant therapy.

Radiation therapy is a cancer treatment that uses high-energy x-rays or other types of radiation to kill cancer cells or keep them from growing. Chemotherapy is a cancer treatment that uses drugs to stop the growth of cancer cells, either by killing the cells or by stopping them from dividing. When chemotherapy is taken by mouth or injected into a vein or muscle, the drugs enter the bloodstream and can reach cancer cells throughout the body (systemic chemotherapy). When chemotherapy is placed directly into the spinal column, an organ, or a body cavity such as the abdomen, the drugs mainly affect cancer cells in those areas (regional chemotherapy). The way the chemotherapy is given depends on the type and stage of the cancer being treated.

Different chemotherapeutic agents are known in art for treating breast cancer. Cytoxic agents used for treating breast cancer include doxorubicin, cyclophosphamide, methotrexate, 5-fluorouracil, mitomycin C, mitoxantrone, paclitaxel, taxane formulations such as by way of example only, Abraxane® (ABI-007), Paclitaxel-Cremophor EL, Paclitaxel poliglumex, and Paclitaxel injectable emulsion (PIE), gemcitabine, docetaxel, capecitabine and epirubicin.

Other chemotherapy against breast cancer includes treatment with one or more of bendamustine, carboplatin (for example, Paraplatin®), carmustine (for example, BCNU®), chlorambucil (for example, Leukeran®), cisplatin (for example, Platinol®), cyclophosphamide injection (for example, Cytoxan®), oral cyclophosphamide (for example, Cytoxan®), dacarbazine (for example, DTIC®), ifosfamide (for example, ifex®), lomustine (for example, CCNU®), mechlorethamine (for example, nitrogen mustard, Mustargen®), melphalan (for example, Alkeran®), procarbazine (for example, Matulane®), bleomycin (for example, Blenoxane®), doxorubicin (for example, Adriamycin®, Rubex®), epirubicin, Idarubicin (for example, Idamycin®), mitoxantrone (for example, Novantrone®), gemcitabine (for example, Gemzar®), oral mercaptopurine (for example, Purinethol®). methotrexate, pentostatin IV (for example, Nipent®), oral thioguanine (for example, Lanvis®), oral etoposide (for example, VP-16, VePesid®, Etopophos)-etoposide IV (for example, VP-16, VePesid®, Etopophos), vinblastine (for example, Velban®), vincristine (for example, Oncovin®), vinorelbine (for example, Navelbine®), dexamethasone (for example, Decadron®), methylprednisolone (for example, Medrol®), and prednisone (for example, Deltasone®).

Hormone therapy is a cancer treatment that removes hormones or blocks their action and stops cancer cells from growing. Hormone therapy with tamoxifen is often given to patients with early stages of breast cancer and those with metastatic breast cancer (cancer that has spread to other parts of the body). Hormone therapy with tamoxifen or estrogens can act on cells all over the body and may increase the chance of developing endometrial cancer. Hormone therapy with an aromatase inhibitor is given to some postmenopausal women who have hormone-dependent breast cancer. Hormone-dependent breast cancer needs the hormone estrogen to grow.

Aromatase inhibitors decrease the body's estrogen by blocking an enzyme called aromatase from turning androgen into estrogen. For the treatment of early stage breast cancer, certain aromatase inhibitors may be used as adjuvant therapy instead of tamoxifen or after 2 or more years of tamoxifen. For the treatment of metastatic breast cancer, aromatase inhibitors are being tested in clinical trials to compare them to hormone therapy with tamoxifen. Examples of aromatse inhibitors currently in use include anastrozole, letrozole and exemestane.

Monoclonal antibody therapy is a cancer treatment that uses antibodies made in the laboratory, from a single type of immune system cell. These antibodies can identify substances on cancer cells or normal substances that may help cancer cells grow. The antibodies attach to the substances and kill the cancer cells, block their growth, or keep them from spreading. Monoclonal antibodies are given by infusion. They may be used alone or to carry drugs, toxins, or radioactive material directly to cancer cells. Monoclonal antibodies are also used in combination with chemotherapy as adjuvant therapy.

Trastuzumab (Herceptin®) is a monoclonal antibody that blocks the effects of the growth factor protein HER2, which transmits growth signals to breast cancer cells.

Trastuzumab leads to clinical responses as a single agent and improves survival when added to chemotherapy for advanced HER2-positive breast cancer. However, some patients do not respond to trastuzumab, and most eventually develop clinical resistance. Mechanisms of intrinsic and acquired trastuzumab resistance are poorly understood. One study which utilized a cell line-based approach to delineate genetic and protein alterations associated with resistance has been reported (D. Tripathy et al Journal of Clinical Oncology, 2005 Vol 23, No 16S, 3121). These researchers studied two HER2-positive breast cancer cell lines (BT474 and SKBR3) that were serially passaged in the presence of trastuzumab until in vitro resistance was documented. Resistant cell lines emerged after 12 months and exhibited a 3-fold more rapid growth rate in the absence of trastuzumab. Following trastuzumab exposure, G₀/G₁ arrest was observed in sensitive compared to resistant cells (84 vs. 68%), with fewer cells in S-phase (3 vs. 14%). Resistant cell lines exhibited fewer changes in gene expression with trastuzumab as well as upregulation of the chemokine receptor CXCR4 and mitotic checkpoint regulators, and downregulation of PTEN compared to sensitive cells.

Thus, additional treatments that may be advantageously combined with the compositions and therapies disclosed herein may further include, without limitation, administration of agents including, but not limited to anastrozole, docetaxel, epirubicin, exemestane, fulvestrant, epothilone A, B or D, goserelin acetate, letrozole, paclitaxel, pamidronate, tamoxifen, toremifene, bevacizumab, or trastuzumab.

As discussed above, provided herein are cancer treatments based on the combination of the compound 2-(4-ethoxyphenyl)-4-methyl-1-(4-sulfamoylphenyl)-pyrrole and lapatinib further in combination with capecitabine. Combinations based on 2-(4-ethoxyphenyl)-4-methyl-1-(4-sulfamoylphenyl)-pyrrole have shown synergistic advantages superior to the effects obtained with celecoxib. These compoundss are described in more detail below.

2-(4-Ethoxyphenyl)-4-methyl-1-(4-sulfamoylphenyl)-pyrrole

2-(4-Ethoxyphenyl)-4-methyl-1-(4-sulfamoylphenyl)-pyrrole is a COX-2 selective inhibitor. U.S. Pat. No. 6,887,893 and RE39,420 describe the preparation of 2-(4-ethoxyphenyl)-4-methyl-1-(4-sulfamoylphenyl)-pyrrole and other chemically-related compounds.

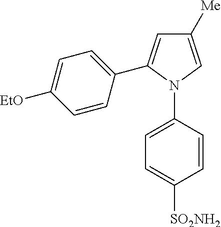

Chemical structure of 2-(4-ethoxyphenyl)-4-methyl-1-(4-sulfamoylphenyl)-pyrrole

### Lapatinib

Lapatinib (Tykerb) is a small molecule and a member of the 4-anilinoquinazoline class of kinase inhibitors. It is present as the monohydrate of the ditosylate salt, with the chemical name N-(3-chloro-4-{[(3-fluorophenyl)methyl]oxy}phenyl)-6-[5-({[2-methylsulfonyl)ethyl]amino}methyl)-2-furanyl]-4-quinazolinamine bis(4-methylbenzenesulfonate) monohydrate. Lapatinib is currently being used to treat advanced or metastatic breast cancer in combination with capecitabine. U.S. Pat. Nos. 6,727,256 and 7,157,466 describe the preparation of lapatinib and other chemically-related compounds. It is a dual inhibitor of EGFR [ErbB1] (Kᵢ=3 nM) and HER2 [ErbB2] (Kᵢ=13 nM) and is indicated for advanced metastatic breast cancer, in combination with capecitabine (Xeloda), in women who have undergone prior treatment with trastuzumab (Herceptin) and taxanes. Lapatinib (1250 mg) is given once daily, one hour before or after food; capecitabine (2000 mg/m²/day) is given twice daily with food.

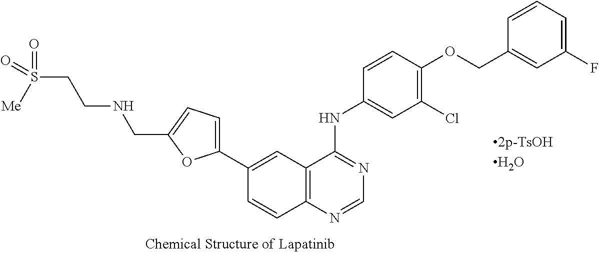

In one embodiment the invention provides a composition comprising a combination of a COX-2 selective inhibitor and an inhibitor of both EGFR [ErbB1] and HER2 [ErbB2] disclosed herein for the treatment and prevention of cancer, tumors, and tumor-related disorders, and neoplastic disease states.

In one embodiment, the inhibitor of both EGFR [ErbB1] and HER2 [ErbB2] is a small molecule compound.

In one embodiment, the inhibitor of both EGFR [ErbB1] and HER2 [ErbB2] is lapatinib. In another embodiment the small molecule compound is selected from the group consisting of: GW2974, gefitinib, AEE788, HKI-272, BIBW-2992 and PKI-166, or their pharmaceutically acceptable salts, solvates, or prodrugs.

Capecitabine

Capecitabine (Xeloda) is an orally-administered chemotherapeutic agent used in the treatment of metastatic breast and colorectal cancer. Capecitabine is a prodrug, that is enzymatically converted to 5-fluorouracil in the tumor, where it inhibits DNA synthesis and slows growth of tumor tissue. The activation of capecitabine follows a pathway with three enzymatic steps and two intermediary metabolites, 5′-deoxy-5-fluorocytidine (5′-DFCR) and 5′-deoxy-5-fluorouridine (5′-DFUR), to form 5-fluorouracil.

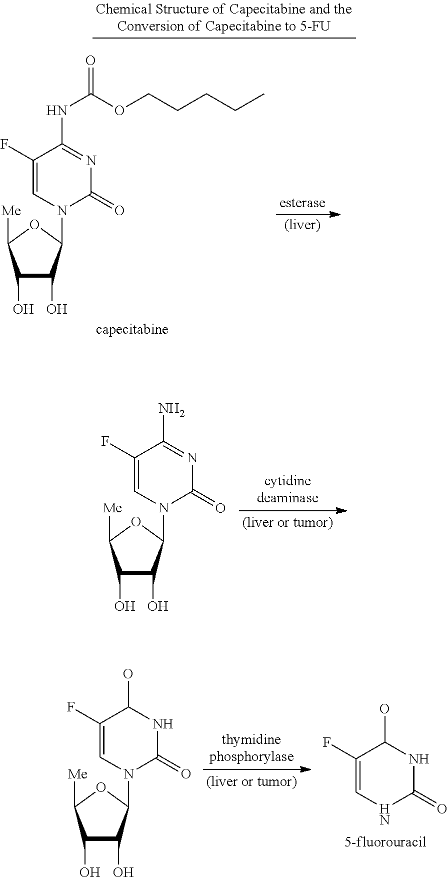

The compositions provided herein may be enantiomerically pure, such as a single enantiomer or a single diastereomer, or be stereoisomeric mixtures, such as a mixture of enantiomers, a racemic mixture, or a diastereomeric mixture, or a polymorph of the active agent. As such, one of skill in the art will recognize that administration of a compound in its (R) form is equivalent, for compounds that undergo epimerization in vivo, to administration of the compound in its (S) form. Conventional techniques for the preparation/isolation of individual enantiomers include chiral synthesis from a suitable optically pure precursor or resolution of the racemate using, for example, chiral chromatography, recrystallization, resolution, diastereomeric salt formation, or derivatization into diastereomeric adducts followed by separation.

When the composition described herein contains an acidic or basic moiety, it may also be provided as a pharmaceutically acceptable salt (See, Berge et al., J. Pharm. Sci. 1977, 66, 1-19; and “Handbook of Pharmaceutical Salts, Properties, and Use,” Stah and Wermuth, Ed.; Wiley-VCH and VHCA, Zurich, 2002).

Suitable acids for use in the preparation of pharmaceutically acceptable salts include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfarnic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-glucosic acid, D-glucuronic acid, L-glutamic acid, α-oxo-glutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts, including, but not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

The composition described herein may also be provided as a prodrug, which is a functional derivative of the 1,2-diphenylpyrrole derivative and/or the inhibitor of both EGFR [ErbB1] and HER2 [ErbB2] and is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis. See Harper, Progress in Drug Research 1962, 4, 221-294; Morozowich et al. in “Design of Biopharmaceutical Properties through Prodrugs and Analogs,” Roche Ed., APHA Acad. Pharm. Sci. 1977; “Bioreversible Carriers in Drug in Drug Design, Theory and Application,” Roche Ed., APHA Acad. Pharm. Sci. 1987; “Design of Prodrugs,” Bundgaard, Elsevier, 1985; Wang et al., Curr. Pharm. Design 1999, 5, 265-287; Pauletti et al., Adv. Drug. Delivery Rev. 1997, 27, 235-256; Mizen et al., Pharm. Biotech. 1998, 11, 345-365; Gaignault et al., Pract. Med. Chem. 1996, 671-696; Asghamejad in “Transport Processes in Pharmaceutical Systems,” Amidon et al., Ed., Marcell Dekker, 185-218, 2000; Balant et al., Eur. J. Drug Metab. Pharmacokinet. 1990, 15, 143-53; Balimane and Sinko, Adv. Drug Delivery Rev. 1999, 39, 183-209; Browne, Clin. Neuropharmacol. 1997, 20, 1-12; Bundgaard, Arch. Pharm. Chem. 1979, 86, 1-39; Bundgaard, Controlled Drug Delivery 1987, 17, 179-96; Bundgaard, Adv. Drug Delivery Rev. 1992, 8, 1-38; Fleisher et al., Adv. Drug Delivery Rev. 1996, 19, 115-130; Fleisher et al., Methods Enzymol. 1985, 112, 360-381; Farquhar et al., J. Pharm. Sci. 1983, 72, 324-325; Freeman et al., J. Chem. Soc., Chem. Commun. 1991, 875-877; Friis and Bundgaard, Eur. J. Pharm. Sci. 1996, 4, 49-59; Gangwar et al., Des. Biopharm. Prop. Prodrugs Analogs, 1977, 409-421; Nathwani and Wood, Drugs 1993, 45, 866-94; Sinhababu and Thakker, Adv. Drug Delivery Rev. 1996, 19, 241-273; Stella et al., Drugs 1985, 29, 455-73; Tan et al., Adv. Drug Delivery Rev. 1999, 39, 117-151; Taylor, Adv. Drug Delivery Rev. 1996, 19, 131-148; Valentino and Borchardt, Drug Discovery Today 1997, 2, 148-155; Wiebe and Knaus, Adv. Drug Delivery Rev. 1999, 39, 63-80; Waller et al., Br. J. Clin. Pharmac. 1989, 28, 497-507.

The combinations presently described herein may also be useful in the treatment of additional disorders in which aberrant expression ligand/receptor interactions or activation or signalling events related to various protein tyrosine kinases are involved. Such disorders may include those of neuronal, glial, astrocytal, hypothalamic, glandular, macrophagal, epithelial, stromal, or blastocoelic nature in which aberrant function, expression, activation or signalling of the erbB tyrosine kinases are involved. In addition, the combinations presented herein may have therapeutic utility in inflammatory, angiogenic and immunologic disorders involving both identified and as yet unidentified tyrosine kinases that are inhibited by the combinations presented herein.

Combinations of therapy for the treatment of non-metastatic adenocarcinoma that may be used in the present disclosure include the use of a composition comprising a combination of 2-(4-ethoxyphenyl)-4-methyl-1-(4-sulfamoylphenyl)-pyrrole and lapatinib along with preoperative bilary tract decompression (patients presenting with obstructive jaundice); surgical resection, including standard resection, extended or radial resection and distal pancreatectomy (tumors of body and tail); adjuvant radiation; antiangiogenic therapy; and chemotherapy.

For the treatment of metastatic adenocarcinoma, a combination therapy consists of a composition comprising a combination of 2-(4-ethoxyphenyl)-4-methyl-1-(4-sulfamoylphenyl)-pyrrole and lapatinib of the present disclosure in combination with continuous treatment of 5-fluorouracil, followed by weekly cisplatin therapy.

Another combination therapy for the treatment of cystic neoplasms is the use of a composition comprising a combination of 2-(4-ethoxyphenyl)-4-methyl-1-(4-sulfamoylphenyl)-pyrrole and lapatinib in combination with gemcitabine.

### Pharmaceutical Compositions

Provided herein are pharmaceutical compositions comprising a combination of a 1,2-diphenylpyrrole derivative and an inhibitor of both EFRG and HER2 [ErbB2] as an active ingredient, or a pharmaceutically acceptable salt, solvate, or prodrug thereof, in a pharmaceutically acceptable vehicle, carrier, diluent, or excipient, or a mixture thereof; and one or more pharmaceutically acceptable excipients or carriers.

Also provided herein are pharmaceutical compositions comprising a combination of a 1,2-diphenylpyrrole derivative and an inhibitor of both EFRG and HER2 [ErbB2] as an active ingredient, or a pharmaceutically acceptable salt, solvate, or prodrug thereof, in a pharmaceutically acceptable vehicle, carrier, diluent, or excipient, or a mixture thereof; and one or more release controlling excipients as described herein. Provided herein are pharmaceutical compositions comprising a combination of a 1,2-diphenylpyrrole derivative and an inhibitor of both EFRG and HER2 [ErbB2] wherein the 1,2-diphenylpyrrole derivative is 2-(4-ethoxyphenyl)-4-methyl-1-(4-sulfamoylphenyl)-pyrrole and the inhibitor of both EFRG and HER2 [ErbB2] is lapatinib as an active ingredient, or a pharmaceutically acceptable salt, solvate, or prodrug thereof, in a pharmaceutically acceptable vehicle, carrier, diluent, or excipient, or a mixture thereof; and one or more release controlling excipients as described herein. Suitable modified release dosage vehicles include, but are not limited to, hydrophilic or hydrophobic matrix devices, water-soluble separating layer coatings, enteric coatings, osmotic devices, multi-particulate devices, and combinations thereof. The pharmaceutical compositions may also comprise non-release controlling excipients.

Provided herein are pharmaceutical compositions in film-coated dosage forms, which comprise a combination of a 1,2-diphenylpyrrole derivative and an inhibitor of both EFRG and HER2 [ErbB2] as an active ingredient, or a pharmaceutically acceptable salt, solvate, or prodrug thereof; and one or more tabletting excipients to form a tablet core using conventional tabletting processes and subsequently coating the core. The tablet cores can be produced using conventional granulation methods, for example wet or dry granulation, with optional comminution of the granules and with subsequent compression and coating. Granulation methods are described, for example, in Voigt, pages 156-69.

Suitable excipients for the production of granules are, for example pulverulent fillers optionally having flow-conditioning properties, for example talcum, silicon dioxide, for example synthetic amorphous anhydrous silicic acid of the Syloid® type (Grace), for example SYLOID 244 FP, microcrystalline cellulose, for example of the Avicel® type (FMC Corp.), for example of the types AVICEL PH101, 102, 105, RC581 or RC591, Emcocel® type (Mendell Corp.) or Elcema® type (Degussa); carbohydrates, such as sugars, sugar alcohols, starches or starch derivatives, for example lactose, dextrose, saccharose, glucose, sorbitol, mannitol, xylitol, potato starch, maize starch, rice starch, wheat starch or amylopectin, tricalcium phosphate, calcium hydrogen phosphate or magnesium trisilicate; binders, such as gelatin, tragacanth, agar, alginic acid, cellulose ethers, for example methylcellulose, carboxymethylcellulose or hydroxypropylmethylcellulose, polyethylene glycols or ethylene oxide homopolymers, especially having a degree of polymerization of approximately from 2.0×10³ to 1.0×10⁵ and an approximate molecular weight of about from 1.0×10⁵ to 5.0×10⁶, for example excipients known by the name Polyox® (Union Carbide), polyvinylpyrrolidone or povidones, especially having a mean molecular weight of approximately 1000 and a degree of polymerization of approximately from about 500 to about 2500, and also agar or gelatin; surface-active substances, for example anionic surfactants of the alkyl sulfate type, for example sodium, potassium or magnesium n-dodecyl sulfate, n-tetradecyl sulfate, n-hexadecyl sulfate or n-octadecyl sulfate, of the alkyl ether sulfate type, for example sodium, potassium or magnesium n-dodecyloxyethyl sulfate, n-tetradecyloxyethyl sulfate, n-hexadecyloxyethyl sulfate or n-octadecyloxyethyl sulfate, or of the alkanesulfonate type, for example sodium, potassium or magnesium n-dodecanesulfonate, n-tetradecanesulfonate, n-hexadecanesulfonate or n-octadecanesulfonate, or non-ionic surfactants of the fatty acid polyhydroxy alcohol ester type, such as sorbitan monolaurate, monooleate, monostearate or monopalmitate, sorbitan tristearate or trioleate, polyoxyethylene adducts of fatty acid polyhydroxy alcohol esters, such as polyoxyethylene sorbitan monolaurate, monooleate, monostearate, monopalmitate, tristearate or trioleate, polyethylene glycol fatty acid esters, such as polyoxyethyl stearate, polyethylene glycol 400 stearate, polyethylene glycol 2000 stearate, especially ethylene oxide/propylene oxide block polymers of the Pluronics® (BWC) or Synperonic® (ICI) type

Further provided herein are pharmaceutical compositions in enteric coated dosage forms, which comprise a combination of a 1,2-diphenylpyrrole derivative and an inhibitor of both EFRG and HER2 [ErbB2] as an active ingredient, or a pharmaceutically acceptable salt, solvate, or prodrug thereof; and one or more release controlling excipients for use in an enteric coated dosage form. Provided herein are pharmaceutical compositions in enteric coated dosage forms comprising a combination of a 1,2-diphenylpyrrole derivative and an inhibitor of both EFRG and HER2 [ErbB2] wherein the 1,2-diphenylpyrrole derivative is 2-(4-ethoxyphenyl)-4-methyl-1-(4-sulfamoylphenyl)-pyrrole and the inhibitor of both EFRG and HER2 [ErbB2] is lapatinib as an active ingredient, or a pharmaceutically acceptable salt, solvate, or prodrug thereof, in a pharmaceutically acceptable vehicle, carrier, diluent, or excipient, or a mixture thereof; and one or more release controlling excipients for use in an enteric coated dosage form. The pharmaceutical compositions may also comprise non-release controlling excipients.

Further provided herein are pharmaceutical compositions in effervescent dosage forms, which comprise a combination of a 1,2-diphenylpyrrole derivative and an inhibitor of both EFRG and HER2 [ErbB2] as an active ingredient, or a pharmaceutically acceptable salt, solvate, or prodrug thereof; and one or more release controlling excipients for use in effervescent dosage forms. Also provided herein are pharmaceutical compositions in effervescent dosage forms comprising a combination of a 1,2-diphenylpyrrole derivative and an inhibitor of both EGFR [ErbB1] and HER2 [ErbB2] wherein the 1,2-diphenylpyrrole derivative is 2-(4-ethoxyphenyl)-4-methyl-1-(4-sulfamoylphenyl)-pyrrole and the inhibitor of both EGFR [ErbB1] and BER2 [ErbB2] is lapatinib as an active ingredient, or a pharmaceutically acceptable salt, solvate, or prodrug thereof, in a pharmaceutically acceptable vehicle, carrier, diluent, or excipient, or a mixture thereof; and one or more release controlling excipients for use in an effervescent dosage forms. The pharmaceutical compositions may also comprise non-release controlling excipients.

Additionally provided are pharmaceutical compositions in a dosage form that has an instant releasing component and at least one delayed releasing component, and is capable of giving a discontinuous release of the compound in the form of at least two consecutive pulses separated in time from 0.1 hour up to 24 hours. The pharmaceutical compositions comprise a combination of a 1,2-diphenylpyrrole derivative and an inhibitor of both EGFR [ErbB1] and HER2 [ErbB2] as an active ingredient, or a pharmaceutically acceptable salt, solvate, or prodrug thereof; and one or more release controlling and non-release controlling excipients, such as those excipients suitable for a disruptable semi-permeable membrane and as swellable substances. Additionally, the invention provides pharmaceutical compositions comprising a combination of a 1,2-diphenylpyrrole derivative and an inhibitor of both EGFR [ErbB1] and HER2 [ErbB2] wherein the 1,2-diphenylpyrrole derivative is 2-(4-ethoxyphenyl)-4-methyl-1-(4-sulfamoylphenye-pyrrole and the inhibitor of both EGFR [ErbB1] and HER2 [ErbB2] is lapatinib as an active ingredient, or a pharmaceutically acceptable salt, solvate, or prodrug thereof; and one or more release controlling and non-release controlling excipients, such as those excipients suitable for a disruptable semi-permeable membrane and as swellable substances.

Provided herein also are pharmaceutical compositions in a dosage form for oral administration to a subject, which comprises a combination of a 1,2-diphenylpyrrole derivative and an inhibitor of both EGFR [ErbB1] and HER2 [ErbB2] as an active ingredient, or a pharmaceutically acceptable salt, solvate, or prodrug thereof; and one or more pharmaceutically acceptable excipients or carriers, enclosed in an intermediate reactive layer comprising a gastric juice-resistant polymeric layered material partially neutralized with alkali and having cation exchange capacity and a gastric juice-resistant outer layer. Additionally, the invention provides pharmaceutical compositions in a dosage form for oral administration to a subject comprising a combination of a 1,2-diphenylpyrrole derivative and an inhibitor of both EGFR [ErbB1] and HER2 [ErbB2] wherein the 1,2-diphenylpyrrole derivative is 2-(4-ethoxyphenyl)-4-methyl-1-(4-sulfamoylphenyl)-pyrrole and the inhibitor of both EGFR [ErbB1] and HER2 [ErbB2] is lapatinib enclosed in an intermediate reactive layer comprising a gastric juice-resistant polymeric layered material partially neutralized with alkali and having cation exchange capacity and a gastric juice-resistant outer layer.

Provided herein are pharmaceutical compositions that comprise a combination of a 1,2-diphenylpyrrole derivative and an inhibitor of both EGFR [ErbB1] and HER2 [ErbB2] as an active ingredient, or a pharmaceutically acceptable salt, solvate, or prodrug thereof, in the form of enteric-coated granules, as delayed-release capsules for oral administration. Also, the invention provides for pharmaceutical compositions that comprise a combination of a 1,2-diphenylpyrrole derivative and an inhibitor of both EGFR [ErbB1] and HER2 [ErbB2] wherein the 1,2-diphenylpyrrole derivative is 2-(4-ethoxyphenyl)-4-methyl-1-(4-sulfamoylphenyl)-pyrrole and the inhibitor of both EGFR [ErbB1] and HER2 [ErbB2] is lapatinib and wherein the quantity of 2-(4-ethoxyphenyl)-4-methyl-1-(4-sulfamoylphenyl)-pyrrole present in the composition is from about 100 mg to about 1200 mg and the quantity of lapatinib present in the composition is from about 500 mg to about 1500 mg wherein both 2-(4-ethoxyphenyl)-4-methyl-1-(4-sulfamoylphenyl)-pyrrole and lapatinib are present as an active ingredient, or a pharmaceutically acceptable salt, solvate, or prodrug thereof, in the form of enteric-coated granules, as delayed-release capsules for oral administration. In additional embodiments, the composition may contain about 100 mg, about 200 mg, about 300 mg, about 400 mg, about 500 mg , about 600 mg, about 700 mg, about 800 mg, about 900 mg, about 1000 mg, about 1100 mg or about 1200 mg of 2-(4-ethoxyphenyl)-4-methyl-1-(4-sulfamoylphenyl)-pyrrole as an active ingredient, or a pharmaceutically acceptable salt, solvate, or prodrug thereof. In additional embodiments, the composition may contain about 500 mg, about 600 mg, about 700 mg, about 800mg, about 900 mg, about 1000 mg, about 1100 mg, about 1200 mg, about 1300 mg, about 1400 mg or about 1500 mg of lapatinib as an active ingredient, or a pharmaceutically acceptable salt, solvate, or prodrug thereof.

The pharmaceutical compositions may further comprise glyceryl monostearate 40-50, hydroxypropyl cellulose, hypromellose, magnesium stearate, methacrylic acid copolymer, sugar spheres, talc, carnauba wax, crospovidone, diacetylated monoglycerides, ethylcellulose, hypromellose phthalate, mannitol, sodium hydroxide, sodium stearyl fumarate, titanium dioxide, yellow ferric oxide, calcium stearate, hydroxypropyl methylcellulose, iron oxide, polysorbate 80, povidone, propylene glycol, sodium carbonate, sodium lauryl sulfate, and triethyl citrate.

The pharmaceutical compositions provided herein may be provided in unit-dosage forms or multiple-dosage forms. Unit-dosage forms, as used herein, refer to physically discrete units suitable for administration to human and animal subjects and packaged individually as is known in the art. Each unit-dose contains a predetermined quantity of the active ingredient(s) sufficient to produce the desired therapeutic effect, in association with the required pharmaceutical carriers or excipients. Examples of unit-dosage forms include ampules, syringes, and individually packaged tablets and capsules. Unit-dosage forms may be administered in fractions or multiples thereof. A multiple-dosage form is a plurality of identical unit-dosage forms packaged in a single container to be administered in segregated unit-dosage form. Examples of multiple-dosage forms include vials, bottles of tablets or capsules, or bottles of pints or gallons.

The compositions provided herein may be administered alone, or in combination with one or more other compounds provided herein, one or more other active ingredients. The pharmaceutical compositions that comprise a compound provided herein may be formulated in various dosage forms for oral, parenteral, buccal, intranasal, epidural, sublingual, pulmonary, local, rectal, transdermal, or topical administration. The pharmaceutical compositions may also be formulated as a modified release dosage form, including delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art (see, Remington: The Science and Practice of Pharmacy, supra; Modified-Release Drug Deliver Technology, Rathbone et al., Eds., Drugs and the Pharmaceutical Science, Marcel Dekker, Inc.: New York, N.Y., 2002; Vol. 126).

The pharmaceutical compositions provided herein may be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

In the case wherein the patient's condition does not improve, upon the doctor's discretion the administration of the combinations may be administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of the combinations may be given continuously or temporarily suspended for a certain length of time (i.e., a “drug holiday”).

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

As described herein, the compositions and methods for using the composition comprising a combination of a 1,2-diphenylpyrrole derivative and an inhibitor of both EGFR [ErbB1] and HER2 [ErbB2], may be formulated without carriers or excipients or may be combined with one or more pharmaceutically acceptable carriers for administration. For example, solvents, diluents and the like, and may be administered orally in such forms as tablets, capsules, dispersible powders, granules, or suspensions containing, for example, from about 0.05 to about 5% of suspending agent, syrups containing, for example, from about 10 to about 50% of sugar, and elixirs containing, for example, from about 20 to about 50% ethanol, and the like. Such pharmaceutical preparations may contain, for example, from about 0.05 up to about 90% of the active ingredient in combination with the carrier, more usually between about 5% and about 60% by weight. Also, the compositions and methods for using the composition comprising a combination of a 1,2-diphenylpyrrole derivative and an inhibitor of both EGFR [ErbB1] and HER2 [ErbB2] wherein the 1,2-diphenylpyrrole derivative is 2-(4-ethoxyphenyl)-4-methyl-1-(4-sulfarnoylphenyl)-pyrrole and the inhibitor of both EGFR [ErbB1] and HER2 [ErbB2] is lapatinib, may be formulated without carriers or excipients or may be combined with one or more pharmaceutically acceptable carriers for administration.

The effective dosage of each active ingredient employed may vary depending on the particular compound employed, the mode of administration and the severity of the condition being treated. The projected daily dosage of the inhibitor of both EGFR [ErbB1] and HER2 [ErbB2] will depend on its potency. Similarly, the dosage of the 1,2-diphenylpyrrole derivative inhibitor used depends on the relative potency of 1,2-diphenylpyrrole derivative inhibitor, compared for example to sulindac. Numerous methods for evaluating and comparing 1,2-diphenylpyrrole derivative inhibitor potency are known to one of skill in the art. In one embodiment, an oral daily dosage of the 1,2-diphenylpyrrole derivative inhibitor is in the range of about 100 to about 1200 mg, and the projected daily dosage of the inhibitor of both EGFR [ErbB1] and HER2 [ErbB2] is in the range of about 1000 to about 1500 mg. In another embodiment, an oral daily dosage of 2-(4-ethoxyphenyl)-4-methyl-1-(4-sulfamoylphenyl)-pyrrole is in the range of about 100 to about 1200 mg, the projected daily dosage of lapatinib is in the range of about 1000 to about 1500 mg and the projected daily dosage of capecitabine is in the range of about 2800 to about 4400 mg. This dosage regimen may be adjusted to provide the optimal therapeutic response. For example, several divided doses may be administered daily or the dose may be proportionally reduced as indicated by the exigencies of the therapeutic situation. The 1,2-diphenylpyrrole derivative inhibitor and the inhibitor of both EGFR [ErbB1] and HER2 [ErbB2] may also be administered as a combined dosage unit, or as separate components. When administered as separate components, each component may be administered at the same time, or at different times during the treatment period.

It is understood, however, that a specific dose level for any particular patient will depend upon a variety of factors such as, for example, decreases in the liver and kidney function.

Treatment dosages generally may be titrated to optimize safety and efficacy. Typically, dosage-effect relationships from in vitro studies initially can provide useful guidance on the proper doses for patient administration. Studies in animal models also generally may be used for guidance regarding effective dosages for treatment of cancers in accordance with the present disclosure. In terms of treatment protocols, it should be appreciated that the dosage to be administered will depend on several factors, including the particular agent that is administered, the route administered, the condition of the particular patient, etc. Determination of these parameters are well within the skill of the art. These considerations, as well as effective formulations and administration procedures are well known in the art and are described in standard textbooks.

### Oral Formulations

Oral formulations containing the active combinations described herein may comprise any conventionally used oral forms, including: tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, syrups, buccal forms, and oral liquids. Capsules may contain mixtures of the active compound(s) with inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, etc. Useful tablet formulations may be made by conventional compression., wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. In some embodiments are surface modifying agents which include nonionic and anionic surface modifying agents. For example, surface modifying agents include, but are not limited to, poloxamer 188, benzalkonium chloride, calcium stearate, cetostearyl alcohol, cetomacrogol emulsifying wax, sorbitan esters, colloidal silicon dioxide, phosphates, sodium dodecylsulfate, magnesium aluminum silicate, and triethanolamine. Oral formulations herein may utilize standard delay or time release formulations to alter the absorption of the active compound(s). The oral formulation may also consist of administering the active ingredient in water or a fruit juice, containing appropriate solubilizers or emulsifiers as needed.

### Oral Administration

As described herein, the combination regimen can be given simultaneously or can be given in a staggered regimen, with a 1,2-diphenylpyrrole derivative being given at a different time during the course of chemotherapy than an inhibitor of both EGFR [ErbB1] and HER2 [ErbB2]. This time differential may range from several minutes, hours, days, weeks, or longer between administration of the two compounds. Therefore, the term combination does not necessarily mean administered at the same time or as a unitary dose, but that each of the components are administered during a desired treatment period. The agents may also be administered by different routes. As is typical for chemotherapeutic regimens, a course of chemotherapy may be repeated several weeks later, and may follow the same timeframe for administration of the two compounds, or may be modified based on patient response.

In other embodiments, the pharmaceutical compositions provided herein may be provided in solid, semisolid, or liquid dosage forms for oral administration. As used herein, oral administration also include buccal, lingual, and sublingual administration. Suitable oral dosage forms include, but are not limited to, tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, and syrups. In addition to the active ingredient(s), the pharmaceutical compositions may contain one or more pharmaceutically acceptable carriers or excipients, including, but not limited to, binders, fillers, diluents, disintegrants, wetting agents, lubricants, glidants, coloring agents, dye-migration inhibitors, sweetening agents, and flavoring agents.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, Panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The binder or filler may be present from about 50 to about 99% by weight in the pharmaceutical compositions provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of disintegrant in the pharmaceutical compositions provided herein varies upon the type of formulation, and is readily discernible to those of ordinary skill in the art. The pharmaceutical compositions provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W.R. Grace Co., Baltimore, Md.) and CAB-O-SIL® (Cabot Co. of Boston, Mass.); and mixtures thereof. The pharmaceutical compositions provided herein may contain about 0.1 to about 5% by weight of a lubricant.

Suitable glidants include colloidal silicon dioxide, CAB-O-SIL® (Cabot Co. of Boston, Mass.), and asbestos-free talc. Coloring agents include any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye. Flavoring agents include natural flavors extracted from plants, such as fruits, and synthetic blends of compounds which produce a pleasant taste sensation, such as peppermint and methyl salicylate. Sweetening agents include sucrose, lactose, mannitol, syrups, glycerin, and artificial sweeteners, such as saccharin and aspartame. Suitable emulsifying agents include gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suspending and dispersing agents include sodium carboxymethylcellulose, pectin, tragacanth, Veegum, acacia, sodium carbomethylcellulose, hydroxypropyl methylcellulose, and polyvinylpyrolidone. Preservatives include glycerin, methyl and propylparaben, benzoic add, sodium benzoate and alcohol. Wetting agents include propylene glycol monostearate, sorbitan monooleate, diethylene glycol monolaurate, and polyoxyethylene lauryl ether. Solvents include glycerin, sorbitol, ethyl alcohol, and syrup. Examples of non-aqueous liquids utilized in emulsions include mineral oil and cottonseed oil. Organic acids include citric and tartaric acid. Sources of carbon dioxide include sodium bicarbonate and sodium carbonate.

It should be understood that many carriers and excipients may serve several functions, even within the same formulation.

In further embodiments, the pharmaceutical compositions provided herein may be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms may be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions provided herein may be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the active ingredient.

In other embodiments, the pharmaceutical compositions provided herein may be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquids or solvent, emulsifying agent, and preservative. Suspensions may include a pharmaceutically acceptable suspending agent and preservative. Aqueous alcoholic solutions may include a pharmaceutically acceptable acetal, such as a di(lower alkyl) acetal of a lower alkyl aldehyde (the term “lower” means an alkyl having between 1 and 6 carbon atoms), e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) provided herein, and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations may further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates.

The pharmaceutical compositions provided herein for oral administration may be also provided in the forms of liposomes, micelles, microspheres, or nanosystems. Miccellar dosage forms can be prepared as described in U.S. Pat. No. 6,350,458.

In other embodiments, the pharmaceutical compositions provided herein may be provided as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in all of the above dosage forms.

The pharmaceutical compositions provided herein may be formulated as immediate or modified release dosage forms, including delayed-, sustained, pulsed-, controlled, targeted-, and programmed-release forms.

In further embodiments, the pharmaceutical compositions provided herein may be co-formulated with other active ingredients which do not impair the desired therapeutic action, or with substances that supplement the desired action, such as other cholinergic agents, other serotoninergic agents, alpha adrenergic agents, CCK_{A} antagonists, 5-HT₃ antagonists, NMDA receptor antagonists, opioids, prokinetics, tachykinins, antalarmin, and Z-338.

### Parenteral Administration

In some embodiments, the pharmaceutical compositions provided herein may be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, and subcutaneous administration.

In other embodiments, the pharmaceutical compositions provided herein may be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions intended for parenteral administration may include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, dimethylacetamide, and dimethylsulfoxide.

Suitable antimicrobial agents or preservatives include, but are not limited to, phenols, cresols, mercurials, benzyl alcohol, chlorobutanol, methyl and propyl p-hydroxybenzates, thimerosal, benzalkonium chloride, benzethonium chloride, methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents include those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, Kans.).

In some embodiments, the pharmaceutical compositions provided herein may be formulated for single or multiple dosage administration. The single dosage formulations are packaged in an ampule, a vial, or a syringe. The multiple dosage parenteral formulations must contain an antimicrobial agent at bacteriostatic or fungistatic concentrations. All parenteral formulations must be sterile, as known and practiced in the art.

In one embodiment, the pharmaceutical compositions are provided as ready-to-use sterile solutions. In another embodiment, the pharmaceutical compositions are provided as sterile dry soluble products, including lyophilized powders and hypodermic tablets, to be reconstituted with a vehicle prior to use. In yet another embodiment, the pharmaceutical compositions are provided as ready-to-use sterile suspensions. In yet another embodiment, the pharmaceutical compositions are provided as sterile dry insoluble products to be reconstituted with a vehicle prior to use. In still another embodiment, the pharmaceutical compositions are provided as ready-to-use sterile emulsions.

The pharmaceutical compositions provided herein may be formulated as immediate or modified release dosage forms, including delayed-, sustained, pulsed-, controlled, targeted-, and programmed-release forms.

The pharmaceutical compositions may be formulated as a suspension, solid, semi-solid, or thixotropic liquid, for administration as an implanted depot. In one embodiment, the pharmaceutical compositions provided herein are dispersed in a solid inner matrix, which is surrounded by an outer polymeric membrane that is insoluble in body fluids but allows the active ingredient in the pharmaceutical compositions diffuse through.

Suitable inner matrixes include polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers, such as hydrogels of esters of acrylic and methacrylic acid, collagen, cross-linked polyvinylalcohol, and cross-linked partially hydrolyzed polyvinyl acetate.

Suitable outer polymeric membranes include polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

### Modified Release

In other embodiments, the pharmaceutical compositions provided herein may be formulated as a modified release dosage form. As used herein, the term “modified release” refers to a dosage form in which the rate or place of release of the active ingredient(s) is different from that of an immediate dosage form when administered by the same route. Modified release dosage forms include delayed-, extended-, prolonged-, sustained-, pulsatile-, controlled-, accelerated- and fast-, targeted-, programmed-release, and gastric retention dosage forms. The pharmaceutical compositions in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphorisim of the active ingredient(s).

Examples of modified release include, but are not limited to, those described in U.S. Pat. Nos.: 3,845,770; 3,916,899; 3,536,809; 3,598,123; 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113,943; 6,197,350; 6,248,363; 6,264,970; 6,267,981; 6,376,461; 6,419,961; 6,589,548; 6,613,358; and 6,699,500.

### 1. Matrix Controlled Release Devices

In some embodiments, the pharmaceutical compositions provided herein in a modified release dosage form may be fabricated using a matrix controlled release device known to those skilled in the art (see, Takada et al in “Encyclopedia of Controlled Drug Delivery,” Vol. 2, Mathiowitz ed., Wiley, 1999).

In one embodiment, the pharmaceutical compositions provided herein in a modified release dosage form is formulated using an erodible matrix device, which is water-swellable, erodible, or soluble polymers, including synthetic polymers, and naturally occurring polymers and derivatives, such as polysaccharides and proteins.

Materials useful in forming an erodible matrix include, but are not limited to, chitin, chitosan, dextran, and pullulan; gum agar, gum arabic, gum karaya, locust bean gum, gum tragacanth, carrageenans, gum ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; and cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethylhydroxy ethylcellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT®, Rohm America, Inc., Piscataway, N.J.); poly(2-hydroxyethyl-methacrylate); polylactides; copolymers of L-glutamic acid and ethyl-L-glutamate; degradable lactic acid-glycolic acid copolymers; poly-D-(−)-3-hydroxybutyric acid; and other acrylic acid derivatives, such as homopolymers and copolymers of butylmethacrylate, methylmethacrylate, ethylmethacrylate, ethylacrylate, (2-dimethylaminoethyl)methacrylate, and (trimethylaminoethyl)methacrylate chloride.

In further embodiments, the pharmaceutical compositions are formulated with a non-erodible matrix device. The active ingredient(s) is dissolved or dispersed in an inert matrix and is released primarily by diffusion through the inert matrix once administered. Materials suitable for use as a non-erodible matrix device included, but are not limited to, insoluble plastics, such as polyethylene, polypropylene, polyisoprene, polyisobutylene, polybutadiene, polymethylmethacrylate, polybutylmethacrylate, chlorinated polyethylene, polyvinylchloride, methyl acrylate-methyl methacrylate copolymers, ethylene-vinylacetate copolymers, ethylene/propylene copolymers, ethylene/ethyl acrylate copolymers, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, polyvinyl chloride, plasticized nylon, plasticized polyethyleneterephthalate, natural rubber, silicone rubbers, polydimethylsiloxanes, silicone carbonate copolymers, and; hydrophilic polymers, such as ethyl cellulose, cellulose acetate, crospovidone, and cross-linked partially hydrolyzed polyvinyl acetate,; and fatty compounds, such as carnauba wax, microcrystalline wax, and triglycerides.

In a matrix controlled release system, the desired release kinetics can be controlled, for example, via the polymer type employed, the polymer viscosity, the particle sizes of the polymer and/or the active ingredient(s), the ratio of the active ingredient(s) versus the polymer, and other excipients or carriers in the compositions.

In other embodiments, the pharmaceutical compositions provided herein in a modified release dosage form may be prepared by methods known to those skilled in the art, including direct compression, dry or wet granulation followed by compression, melt-granulation followed by compression.

### 2. Osmotic Controlled Release Devices

In some embodiments, the pharmaceutical compositions provided herein in a modified release dosage form may be fabricated using an osmotic controlled release device, including one-chamber system, two-chamber system, asymmetric membrane technology (AMT), and extruding core system (ECS). In general, such devices have at least two components: (a) the core which contains the active ingredient(s); and (b) a semipermeable membrane with at least one delivery port, which encapsulates the core. The semipermeable membrane controls the influx of water to the core from an aqueous environment of use so as to cause drug release by extrusion through the delivery port(s).

In addition to the active ingredient(s), the core of the osmotic device optionally includes an osmotic agent, which creates a driving force for transport of water from the environment of use into the core of the device. One class of osmotic agents water-swellable hydrophilic polymers, which are also referred to as “osmopolymers” and “hydrogels,” including, but not limited to, hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP), crosslinked PVP, polyvinyl alcohol (PVA), PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

The other class of osmotic agents are osmogens, which are capable of imbibing water to affect an osmotic pressure gradient across the barrier of the surrounding coating. Suitable osmogens include, but are not limited to, inorganic salts, such as magnesium sulfate, magnesium chloride, calcium chloride, sodium chloride, lithium chloride, potassium sulfate, potassium phosphates, sodium carbonate, sodium sulfite, lithium sulfate, potassium chloride, and sodium sulfate; sugars, such as dextrose, fructose, glucose, inositol, lactose, maltose, mannitol, raffinose, sorbitol, sucrose, trehalose, and xylitol,; organic acids, such as ascorbic acid, benzoic acid, fumaric acid, citric acid, maleic acid, sebacic acid, sorbic acid, adipic acid, edetic acid, glutamic acid, p-tolunesulfonic acid, succinic acid, and tartaric acid; urea; and mixtures thereof.

Osmotic agents of different dissolution rates may be employed to influence how rapidly the active ingredient(s) is initially delivered from the dosage form. For example, amorphous sugars, such as Mannogeme EZ (SPI Pharma, Lewes, Del.) can be used to provide faster delivery during the first couple of hours to promptly produce the desired therapeutic effect, and gradually and continually release of the remaining amount to maintain the desired level of therapeutic or prophylactic effect over an extended period of time. In this case, the active ingredient(s) is released at such a rate to replace the amount of the active ingredient metabolized and excreted.

The core may also include a wide variety of other excipients and carriers as described herein to enhance the performance of the dosage form or to promote stability or processing.

Materials useful in forming the semi-permeable membrane include various grades of acrylics, vinyls, ethers, polyamides, polyesters, and cellulosic derivatives that are water-permeable and water-insoluble at physiologically relevant pHs, or are susceptible to being rendered water-insoluble by chemical alteration, such as crosslinking. Examples of suitable polymers useful in forming the coating, include plasticized, unplasticized, and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxylated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes, and synthetic waxes.

Semi-permeable membrane may also be a hydrophobic microporous membrane, wherein the pores are substantially filled with a gas and are not wetted by the aqueous medium but are permeable to water vapor, as disclosed in U.S. Pat. No. 5,798,119. Such hydrophobic but water-vapor permeable membrane are typically composed of hydrophobic polymers such as polyalkenes, polyethylene, polypropylene, polytetrafluoroethylene, polyacrylic acid derivatives, polyethers, polysulfones, polyethersulfones, polystyrenes, polyvinyl halides, polyvinylidene fluoride, polyvinyl esters and ethers, natural waxes, and synthetic waxes.

The delivery port(s) on the semi-permeable membrane may be formed post-coating by mechanical or laser drilling. Delivery port(s) may also be formed in situ by erosion of a plug of water-soluble material or by rupture of a thinner portion of the membrane over an indentation in the core. In addition, delivery ports may be formed during coating process, as in the case of asymmetric membrane coatings of the type disclosed in U.S. Pat. Nos. 5,612,059 and 5,698,220.

The total amount of the active ingredient(s) released and the release rate can substantially by modulated via the thickness and porosity of the semi-permeable membrane, the composition of the core, and the number, size, and position of the delivery ports.

The pharmaceutical compositions in an osmotic controlled-release dosage form may further comprise additional conventional excipients or carriers as described herein to promote performance or processing of the formulation.

The osmotic controlled-release dosage forms can be prepared according to conventional methods and techniques known to those skilled in the art (see, Remington: The Science and Practice of Pharmacy, supra; Santus and Baker, J. Controlled Release 1995, 35, 1-21; Verma et al., Drug Development and Industrial Pharmacy 2000, 26, 695-708; Verma et al., J. Controlled Release 2002, 79, 7-27).

In other embodiments, the pharmaceutical compositions provided herein are formulated as AMT controlled-release dosage form, which comprises an asymmetric osmotic membrane that coats a core comprising the active ingredient(s) and other pharmaceutically acceptable excipients or carriers. See, U.S. Pat. No. 5,612,059 and WO 2002/17918. The AMT controlled-release dosage forms can be prepared according to conventional methods and techniques known to those skilled in the art, including direct compression, dry granulation, wet granulation, and a dip-coating method.

In certain embodiments, the pharmaceutical compositions provided herein are formulated as ESC controlled-release dosage form, which comprises an osmotic membrane that coats a core comprising the active ingredient(s), a hydroxylethyl cellulose, and other pharmaceutically acceptable excipients or carriers.

### 3. Multiparticulate Controlled Release Devices

In some embodiments, the pharmaceutical compositions provided herein in a modified release dosage form may be fabricated as a rnultiparticulate controlled release device, which comprises a multiplicity of particles, granules, or pellets, ranging from about 10 μm to about 3 mm, about 50 μm to about 2.5 mm, or from about 100 μm to about 1 mm in diameter. Such multiparticulates may be made by the processes know to those skilled in the art, including wet-and dry-granulation, extrusion/spheronization, roller-compaction, melt-congealing, and by spray-coating seed cores. See, for example, Multiparticulate Oral Drug Delivery; Marcel Dekker: 1994; and Pharmaceutical Pelletization Technology; Marcel Dekker: 1989.

Other excipients or carriers as described herein may be blended with the pharmaceutical compositions to aid in processing and forming the multiparticulates. The resulting particles may themselves constitute the multipartieulate device or may be coated by various film-forming materials, such as enteric polymers, water-swellable, and water-soluble polymers. The multiparticulates can be further processed as a capsule or a tablet.

### 4. Targeted Delivery

In some embodiments, the pharmaceutical compositions provided herein may also be formulated to be targeted to a particular tissue, receptor, or other area of the body of the subject to be treated, including liposome-, resealed erythrocyte-, and antibody-based delivery systems. Examples include, but are not limited to, U.S. Pat. Nos. 6,316,652; 6,274,552; 6,271,359; 6,253,872; 6,139,865; 6,131,570; 6,120,751; 6,071,495; 6,060,082; 6,048,736; 6,039,975; 6,004,534; 5,985,307; 5,972,366; 5,900,252; 5,840,674; 5,759,542; and 5,709,874, all of which are incorporated herein by their entirety.

### Immediate Release

In some embodiments, the pharmaceutical compositions provided herein in an immediate release dosage form are capable of releasing not less than 75% of the therapeutically active ingredient or combination and/or meet the disintegration or dissolution requirements for immediate release tablets of the particular therapeutic agents or combination included in the tablet core, as set forth in USP XXII, 1990 (The United States Pharmacopeia.)

### Topical Administration

In other embodiments, the pharmaceutical compositions provided herein may be administered topically to the skin, orifices, or mucosa. The topical administration, as used herein, include (intra)dermal, conjuctival, intracorneal, intraocular, ophthalmic, auricular, transdermal, nasal, vaginal, uretheral, respiratory, and rectal administration.

In further embodiments, the pharmaceutical compositions provided herein may be formulated in any dosage forms that are suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, dermal patches. The topical formulation of the pharmaceutical compositions provided herein may also comprise liposomes, micelles, microspheres, nanosystems, and mixtures thereof.

Pharmaceutically acceptable carriers and excipients suitable for use in the topical formulations provided herein include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryopretectants, lyoprotectants, thickening agents, and inert gases.

In some embodiments, the pharmaceutical compositions may also be administered topically by electroporation, iontophoresis, phonophoresis, sonophoresis and microneedle or needle-free injection, such as POWDERJECT™ (Chiron Corp., Emeryville, Calif.), and BIOJECT™ (Bioject Medical Technologies Inc., Tualatin, Oreg.).

The pharmaceutical compositions provided herein may be provided in the forms of ointments, creams, and gels. Suitable ointment vehicles include oleaginous or hydrocarbon vehicles, including such as lard, benzoinated lard, olive oil, cottonseed oil, and other oils, white petrolatum; emulsifiable or absorption vehicles, such as hydrophilic petrolatum, hydroxystearin sulfate, and anhydrous lanolin; water-removable vehicles, such as hydrophilic ointment; water-soluble ointment vehicles, including polyethylene glycols of varying molecular weight; emulsion vehicles, either water-in-oil (W/O) emulsions or oil-in-water (O/W) emulsions, including cetyl alcohol, glyceryl monostearate, lanolin, and stearic acid (see, Remington: The Science and Practice of Pharmacy, supra). These vehicles are emollient but generally require addition of antioxidants and preservatives.

Suitable cream base can be oil-in-water or water-in-oil. Cream vehicles may be water-washable, and contain an oil phase, an emulsifier, and an aqueous phase. The oil phase is also called the “internal” phase, which is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation may be a nonionic, anionic, cationic, or amphoteric surfactant.

Gels are semisolid, suspension-type systems. Single-phase gels contain organic macromolecules distributed substantially uniformly throughout the liquid carrier. Suitable gelling agents include crosslinked acrylic acid polymers, such as carbomers, carboxypolyalkylenes, Carbopol®; hydrophilic polymers, such as polyethylene oxides, polyoxyethylene-polyoxypropylene copolymers, and polyvinylalcohol; cellulosic polymers, such as hydroxypropyl cellulose, hydroxyethyl cellulose, hydroxypropyl methylcellulose, hydroxypropyl methylcellulose phthalate, and methylcellulose; gums, such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

The pharmaceutical compositions provided herein may be administered rectally, urethrally, vaginally, or perivaginally in the forms of suppositories, pessaries, bougies, poultices or cataplasm, pastes, powders, dressings, creams, plasters, contraceptives, ointments, solutions, emulsions, suspensions, tampons, gels, foams, sprays, or enemas. These dosage forms can be manufactured using conventional processes as described in Remington: The Science and Practice of Pharmacy, supra.

Rectal, urethral, and vaginal suppositories are solid bodies for insertion into body orifices, which are solid at ordinary temperatures but melt or soften at body temperature to release the active ingredient(s) inside the orifices. Pharmaceutically acceptable carriers utilized in rectal and vaginal suppositories include bases or vehicles, such as stiffening agents, which produce a melting point in the proximity of body temperature, when formulated with the pharmaceutical compositions provided herein; and antioxidants as described herein, including bisulfite and sodium metabisulfite. Suitable vehicles include, but are not limited to, cocoa butter (theobroma oil), glycerin-gelatin, carbowax (polyoxyethylene glycol), spermaceti, paraffin, white and yellow wax, and appropriate mixtures of mono-, di- and triglycerides of fatty acids, hydrogels, such as polyvinyl alcohol, hydroxyethyl methacrylate, polyacrylic acid; glycerinated gelatin. Combinations of the various vehicles may be used. Rectal and vaginal suppositories may be prepared by the compressed method or molding. The typical weight of a rectal and vaginal suppository is about 2 to about 3 g.

The pharmaceutical compositions provided herein may be administered ophthalmically in the forms of solutions, suspensions, ointments, emulsions, gel-forming solutions, powders for solutions, gels, ocular inserts, and implants.

The pharmaceutical compositions provided herein may be administered intranasally or by inhalation to the respiratory tract. The pharmaceutical compositions may be provided in the form of an aerosol or solution for delivery using a pressurized container, pump, spray, atomizer, such as an atomizer using electrohydrodynamics to produce a fine mist, or nebulizer, alone or in combination with a suitable propellant, such as 1,1,1,2-tetrafluoroethane or 1,1,1,2,3,3,3-heptafluoropropane. The pharmaceutical compositions may also be provided as a dry powder for insufflation, alone or in combination with an inert carrier such as lactose or phospholipids; and nasal drops. For intranasal use, the powder may comprise a bioadhesive agent, including chitosan or cyclodextrin.

Solutions or suspensions for use in a pressurized container, pump, spray, atomizer, or nebulizer may be formulated to contain ethanol, aqueous ethanol, or a suitable alternative agent for dispersing, solubilizing, or extending release of the active ingredient provided herein, a propellant as solvent; and/or an surfactant, such as sorbitan trioleate, oleic acid, or an oligolactic acid.

In another embodiment, the pharmaceutical compositions provided herein may be micronized to a size suitable for delivery by inhalation, such as about 50 micrometers or less, or about 10 micrometers or less. Particles of such sizes may be prepared using a comminuting method known to those skilled in the art, such as spiral jet milling, fluid bed jet milling, supercritical fluid processing to form nanoparticles, high pressure homogenization, or spray drying.

Capsules, blisters and cartridges for use in an inhaler or insufflator may be formulated to contain a powder mix of the pharmaceutical compositions provided herein; a suitable powder base, such as lactose or starch; and a performance modifier, such as 1-leucine, mannitol, or magnesium stearate. The lactose may be anhydrous or in the form of the monohydrate. Other suitable excipients include dextran, glucose, maltose, sorbitol, xylitol, fructose, sucrose, and trehalose. The pharmaceutical compositions provided herein for inhaled/intranasal administration may further comprise a suitable flavor, such as menthol and levomenthol, or sweeteners, such as saccharin or saccharin sodium.

In one embodiment, the pharmaceutical compositions provided herein for topical administration may be formulated to be immediate release or modified release, including delayed-, sustained-, pulsed-, controlled-, targeted, and programmed release.

### EXAMPLES

### Example 1

### Synthesis of 2-(4-ethoxyphenyl)-4-methyl-1-(4-sulfamoylphenyl)-pyrrole

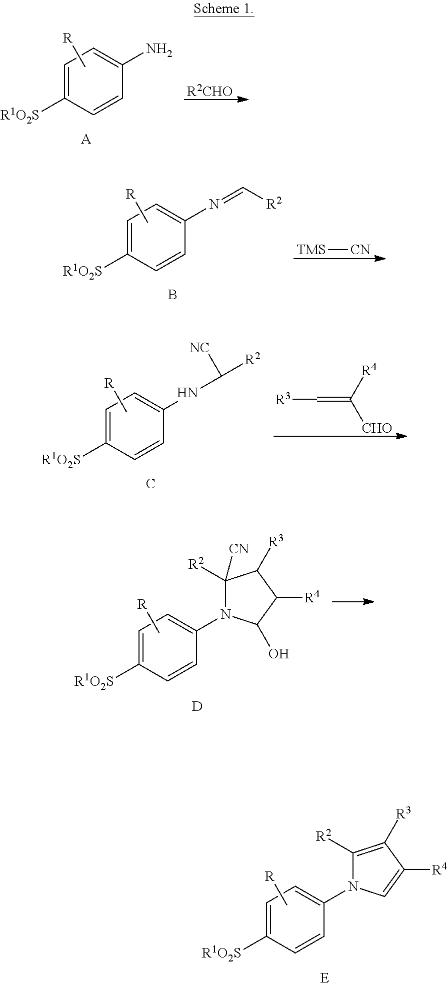

Substituted benzaldehyde undergoes dehydration condensation by reaction with aniline compound A in an inert solvent at a temperature of between 5° C. to 200° C. to give aldimine compound B. Trimethylsilyl cyanide is then reacted with aldimine compound B in the presence of a Lewis acid to afford anilinonitrile C. An α,β-unsaturated aldehyde is then reacted with anilinonitrile C to afford compound D which then undergoes dehydration and dehydrogencyanation under basic conditions in a modification of the method described in Ann. Chem. 589, 176 (1954).

### Example 2

### Synthesis of Lapatinib

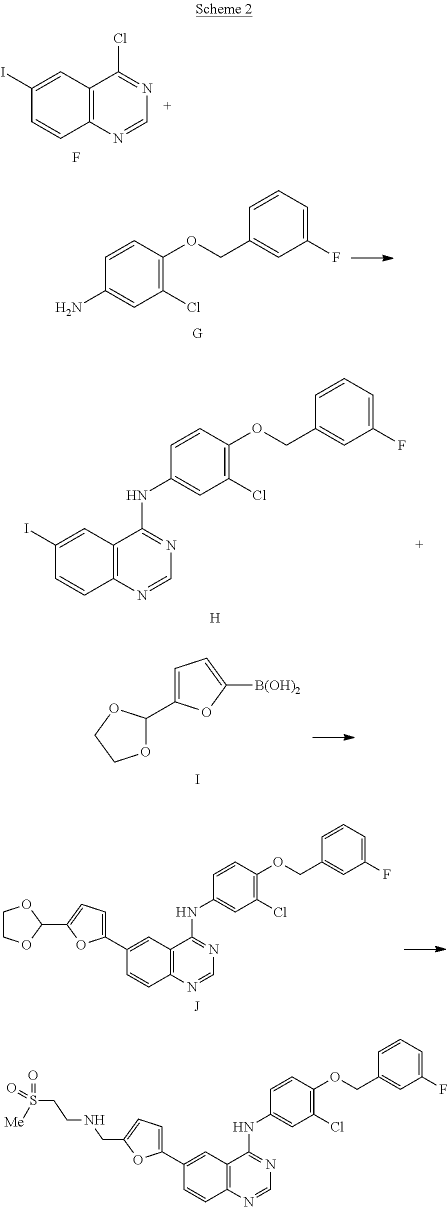

Starting compound F is condensed with aniline G under basic conditions such as K₂CO₃ in DMF as solvent. Iodoquinazoline H is subjected to a palladium (0) catalyzed cross coupling reaction with furan boronic acid I to afford heterocycle J. Deprotection of J followed by reductive amination with 2-methanesulphonylethylamine will give lapatinib.

### Example 3

### Synthesis of Capecitabine

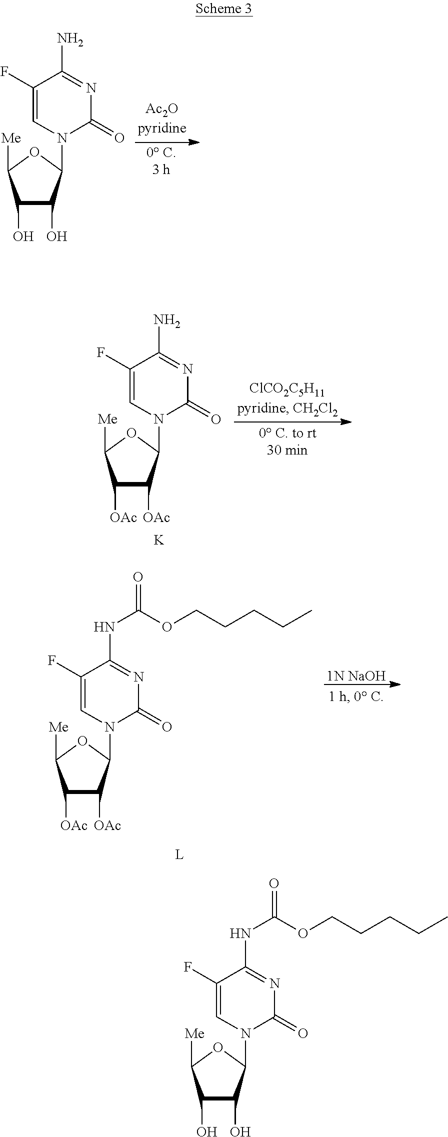

5′-Deoxy-5-fluorocytidine is dissolved in pyridine and reacted with acetic anhydride to afford diacetyl compound K. Reaction of K with n-pentylchloroformate provided diacylcapecitabine L. Hydrolysis of the acetyl groups in compound L is performed by treating the compound with aqueous NaOH for 1 h in an ice bath to give capecitabine.

### Example 4

### Pharmacokinetics and Metabolism of 2-(4-ethoxyphenyl)-4-methyl-1-(4-sulfamoylphenyl)-pyrrole

Orally administered 2-(4-ethoxyphenyl)-4-methyl-1-(4-sulfamoylphenyl)-pyrrole is rapidly absorbed in all species examined (mice, rats, dogs, and monkeys). Peak plasma concentrations were achieved between 1 and 3 hours after a dose of 5 mg/kg. The elimination half life (t_{1/2}) was 4-5 hours in rodents and dogs, and approximately 2 hours in monkeys. Oral availability was greatest in rodent, and was reduced in dogs and monkeys (59 and 34% respectively). Pharmacokinetics in human subjects demonstrated a linear dose exposure relationship from doses of 2 mg to 800 mg given orally. The half-life in human subjects is 15-18 hours.

### Example 5

### Toxicology of 2-(4-ethoxyphenyl)-4-methyl-1-(4-sulfamoylphenyl)-pyrrole

Toxicological evaluation of 2-(4-ethoxyphenyl)-4-methyl-1-(4-sulfamoylphenyl)-pyrrole in mice, rats, dogs and monkeys revealed expected findings related to inhibition of cyclooxygenase and consistent with animal safety observations with other COX-2 selective inhibitors. In single dose studies, the minimum lethal dose of 2-(4-ethoxyphenyl)-4-methyl-1-(4-sulfamoylphenyl)-pyrrole was 600 mg/kg in rats and >2000 mg/kg in dogs. An endoscopy study conducted in human subjects demonstrated no increase in gastric or duodenal toxicity compared to placebo.

### Example 6

### Biological Evaluation

### COX-2 Selective Inhibitors

### SK-BR-3 Model:

Mice are injected subcutaneously in the left paw (1×10⁶ tumor cells suspended in 30% Matrigel) and tumor volume is evaluated using a phlethysmometer twice a week for 30-60 days. Implantation of human breast cancer cells (SK-BR-3) into nude mice produces tumors that will reach 0.6-2 ml between 30-50 days. Blood is drawn twice during the experiment in a 24 h protocol to assess plasma concentration and total exposure by AUC analysis. The data is expressed as the mean+/−SEM. Student's and Mann-Whitney tests are used to assess differences between means using the InStat software package.

A. Mice injected with SK-BR-3 cancer cells are treated with cytoxin i.p at doses of 50 mg/kg on days 5, 7 and 9 in the presence or absence of a composition comprising a combination of 2-(4-ethoxyphenyl)-4-methyl-1-(4-sulfamoylphenyl)-pyrrole with lapatinib in the diet. The efficacy of both agents are determined by measuring tumor volume. The results from these studies may demonstrate that a composition comprising a combination of 2-(4-ethoxyphenyl)-4-methyl-1-(4-sulfamoylphenyl)-pyrrole with lapatinib administered in the diet to tumor bearing mice can delay the growth of tumors and metastasis when a ministered as sole therapy.

B. In a second assay, mice are injected with SK-BR-3 cancer cells are then treated with 5-FU on days 12 through 15. Mice injected with SK-BR-3 cancer cells are treated with 5-FU i.p at doses of 50 mg/kg on days 12, 13, 14, and 15 in the presence or absence of a composition comprising a combination of 2-(4-ethoxyphenyl)-4-methyl-1-(4-sulfamoylphenyl)-pyrrole with lapatinib in the diet. The efficacy of both agents are determined by measuring tumor volume. Treatment using the composition may reduce tumor volume by up to 70%. In the same assay, 5-FU decreases tumor volume by 61%. Further, the composition and 5-FU may decrease tumor volume by 83%.

C. In a third assay, mice injected with SK-BR-3 breast cancer cells are treated with 5-FU i.p 50 mg/kg on days 14 through 17 in the presence or absence of a composition comprising a combination of 2-(4-ethoxyphenyl)-4-methyl-1-(4-sulfamoylphenyl)-pyrrole with lapatinib (1600 ppm) and valdecoxib (160 ppm) in the diet. The efficacy of both agents are determined by measuring tumor volume. Treatment with 5-FU may result in a 35% reduction in tumor volume. Treatment with the composition and valdecoxib may reduce tumor volume by 52% and 69%, respectively. In the same assay, the combination of 5-FU and the composition may decrease tumor volume by 72% while the combination of 5-FU and valdecoxib may decrease tumor volume by 74%.

### Example 7

### In Vitro Inhibition of EGFR [ErbB11 Kinase Activity

The in vitro activity of the combinations described herein in inhibiting the receptor tyrosine kinase may be determined by the following procedure. The in vitro activity of the combinations of the present disclosure can be determined by the amount of inhibition of the phosphorylation of an exogenous substrate (e.g., Lys₃—Gastrin or polyGluTyr (4:1) random copolymer (Posner et al., J. Biol. Chem., 1992, 267 (29), 20638-472)) on tyrosine by epidermal growth factor receptor kinase by a test compound relative to a control. Affinity purified, soluble human EGF receptor (96 ng) is obtained according to the procedure in G. N. Gill, W. Weber, Methods in Enzymology, 1987, 146, 82-8 from A431 cells (American Type Culture Collection, Rockville, Md.) and preincubated in a microfuge tube with EGF (2 μg/ml) in phosphorylation buffer+vanadate (PBV: 50 mM HEPES, pH 7.4; 125 mM NaCl; 24 mM MgCl₂; 100 μM sodium orthovanadate), in a total volume of 10 μl, for 20-30 minutes at room temperature. The composition comprising a combination of 2-(4-ethoxyphenyl)-4-methyl-1-(4-sulfamoylphenyl)-pyrrole with lapatinib, dissolved in dimethylsulfoxide (DMSO), is diluted in PBV, and 10 μl is mixed with the EGF receptor/EGF mix, and incubated for 10-30 minutes at 30° C. The phosphorylation reaction is initiated by addition of 20 μl ³³P-ATP/substrate mix (120 μM Lys₃-Gastrin (sequence in single letter code for amino acids, KKKGPWLEEEEEAYGWLDF), 50 mM Hepes pH 7.4, 40 μM ATP, 2 pCi .gamma.-[³³ P]-ATP) to the EGFr/EGF mix and incubated for 20 minutes at room temperature. The reaction is stopped by addition of 10 μl stop solution (0.5 M EDTA, pH 8; 2 mM ATP) and 6 μl 2N HCl. The tubes are centrifuged at 14,000 RPM, 4° C., for 10 minutes. 35 μl of supernatant from each tube is pipetted onto a 2.5 cm circle of Whatman P81 paper, bulk washed four times in 5% acetic acid, 1 liter per wash, and then air dried. This results in the binding of substrate to the paper with loss of free ATP on washing. The [³³P] incorporated is measured by liquid scintillation counting. Incorporation in the absence of substrate (e.g., lys₃-gastrin) is subtracted from all values as a background and percent inhibition is calculated relative to controls without the composition present. Such assays, carried out with a range of doses of test combinations, allow the determination of an approximate IC₅₀ value for the in vitro inhibition of EGFR [ErbB1] kinase activity. Other methods for determining the activity of the combinations presented herein are described in U.S. Pat. No. 5,747,498, the disclosure of which is incorporated herein.

### Example 8

### In Vitro Inhibition of HER2 [ErbB2] Kinase Activity

The in vitro activity of the combinations described herein in inhibiting the HER2 [ErbB2] receptor tyrosine kinase may be determined by the following procedure. The HER2 [ErbB2] recombinant intracellular domain (amino acids 675-1255) is expressed in baculovirus-infected Sf9 cells as a glutathione S-transferase fusion protein. The protein is purified by affinity chromatography on glutathione Sepharose beads for use in the assay. Nunc MaxiSorp 96-well plates were coated by incubation overnight at 37° C. with 100 μl/well of 0.25 mg/ml poly(Glu:Tyr, 4:1), (PGT; Sigma Chemical Co.) in PBS. Excess PGT is removed by aspiration and the plate is washed 3 times with wash buffer (0.1% Tween 20 in PBS). The kinase reaction is performed in 50 μl of 50 mM HEPES (pH 7.4) containing 125 mM sodium chloride, 10 mM magnesium chloride, 0.1 mM sodium orthovanadate, 1 mM ATP, and about 15 ng of recombinant protein. The test composition in DMSO is added; the final DMSO concentration is 2.5%. Phosphorylation is initiated by addition of ATP and allowed to proceeded for 6 min at room temperature, with constant shaking. The kinase reaction is terminated by aspiration of the reaction mixture and washing four times with wash buffer. Phosphorylated PGT is measured after a 25-min incubation with 50 μl/well HRP conjugated-PY54 (Oncogene Science Inc. Pharmaceuticals, Uniondale, N.Y.) antiphosphotyrosine antibody, diluted to 0.2 μg/ml in blocking buffer (3% BSA, 0.05% Tween 20 in PBS). Antibody is removed by aspiration and the plate is washed four times with wash buffer. The colorimetric signal is developed by addition of 50 μl/well Tetramethylbenzidine Microwell Peroxidase Substrate (Kirkegaard and Perry Labs, Gaithersburg, Md.) and stopped by the addition of 50 μl/well 0.09 M sulfuric acid. The phosphotyrosine product formed is estimated by measurement of absorbance at 450 nm. The signal for controls is typically A0.6-1.2, with essentially no background in wells without ATP, kinase protein, or PGT, and is proportional to the time of incubation for 6 min.

### Example 9

### Pharmaceutical Compositions and Dosage Forms

Dosage formulations comprising pharmaceutical excipients and carriers and a pharmaceutical composition comprising a combination of lapatinib (A) and 2-(4-ethoxyphenyl)-4-methyl-1-(4-sulfamoylphenyl)-pyrrole (B) include:

| | Amount of A per | |
|---|---|---|
| Combination | tablet (mg) | Amount of B per tablet (mg) |
| A/B | 500 | 100, 200, 300, 400, 500, 600, |
| | | 700, 800, 900, 1000, 1100, 1200 |
| A/B | 600 | 100, 200, 300, 400, 500, 600, |
| | | 700, 800, 900, 1000, 1100, 1200 |
| A/B | 700 | 100, 200, 300, 400, 500, 600, |
| | | 700, 800, 900, 1000, 1100, 1200 |
| A/B | 800 | 100, 200, 300, 400, 500, 600, |
| | | 700, 800, 900, 1000, 1100, 1200 |
| A/B | 900 | 100, 200, 300, 400, 500, 600, |
| | | 700, 800, 900, 1000, 1100, 1200 |
| A/B | 1000 | 100, 200, 300, 400, 500, 600, |
| | | 700, 800, 900, 1000, 1100, 1200 |
| A/B | 1100 | 100, 200, 300, 400, 500, 600, |
| | | 700, 800, 900, 1000, 1100, 1200 |
| A/B | 1200 | 100, 200, 300, 400, 500, 600, |
| | | 700, 800, 900, 1000, 1100, 1200 |
| A/B | 1300 | 100, 200, 300, 400, 500, 600, |
| | | 700, 800, 900, 1000, 1100, 1200 |
| A/B | 1400 | 100, 200, 300, 400, 500, 600, |
| | | 700, 800, 900, 1000, 1100, 1200 |
| A/B | 1500 | 100, 200, 300, 400, 500, 600, |
| | | 700, 800, 900, 1000, 1100, 1200 |

Dosage formulations described herein, including the formulations set forth in the above table, may be administered in a single fixed dose comprising a combination of 2-(4-ethoxyphenyl)-4-methyl-1-(4-sulfamoylphenyl)-pyrrole and lapatinib or as a separate administration of a single dose of 2-(4-ethoxyphenyl)-4-methyl-1-(4-sulfamoylphenyl)-pyrrole and a single dose of lapatinib.

### Example 10

### Treatment of Breast Cancer

A method for treating a subject having breast cancer comprising administering to the subject a therapeutically effective amount of a combination comprising 2-(4-ethoxyphenyl)-4-methyl-1-(4-sulfamoylphenyl)-pyrrole and lapatinib or their respective pharmaceutically acceptable salt, solvate or prodrug is contemplated. Combined treatment with lapatinib and 2-(4-ethoxyphenyl)-4-methyl-1-(4-sulfamoylphenyl)-pyrrole is expected to achieve increased tumor inhibition compared with lapatinib administered as a single agent. Combinations based on 2-(4-ethoxyphenyl)-4-methyl-1-(4-sulfamoylphenyl)-pyrrole have shown synergistic advantages superior to the effects obtained with celecoxib.

### Example 11

### 2-(4-Ethoxyphenyl)-4-methyl-1-(4-sulfamoylphenyl)pyrrole in Combination with Lapatinib and Capecitabine in the Treatment of Patients with HER2 [ErbB2] Positive Breast Cancer who have Failed Herceptin

Women with HER2 [ErbB2] positive, advanced metastatic breast cancer that has progressed after treatment with regimens that include anthracyclines, taxanes and trastuzumab are treated with a combination thearpy of 2-(4-ethoxyphenyl)-4-methyl-1-(4-sulfamoylphenyl)pyrrole (one oral dose of 400 mg/day continuously) plus lapatinib (one oral dose of 1200 mg/day continuously) plus capecitabine (one oral dose of 1700 mg every 12 hours for days 1-14 of a 21 day cycle).

1. A method for treating a subject having cancer, comprising administering to the subject, a therapeutically effective amount of a combination comprising a 1,2-diphenylpyrrole derivative and a HER2 [ErbB2] inhibitor or their respective pharmaceutically acceptable salt, solvate, polymorph or prodrug.

2. A method for treating a subject having cancer, comprising administering to the subject, a therapeutically effective amount of a combination comprising a 1,2-diphenylpyrrole derivative and an inhibitor of both EGFR [Erb1] and HER2 [ErbB2] or their respective pharmaceutically acceptable salt, solvate, polymorph or prodrug.

3. The method of claim 2 wherein the 1,2-diphenylpyrrole derivative has the following formula: 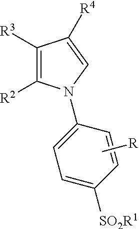
wherein:
R is a hydrogen atom, a halogen atom or an alkyl group having from 1 to 6 carbon atoms;
R¹ is an alkyl group having from 1 to 6 carbon atoms or an amino group;
R² is a phenyl group which is unsubstituted or is substituted by at least one substituent selected from the group consisting of substituents α and substituents β;
R³ is a hydrogen atom, a halogen atom or an alkyl group which has from 1 to 6 carbon atoms and which is unsubstituted or is substituted by at least one substituent selected from the group consisting of a hydroxy group, a halogen atom, an alkoxy group having from 1 to 6 carbon atoms and an atkylthio group having from 1 to 6 carbon atoms;
R⁴ is a hydrogen atom; an alkyl group which has from 1 to 6 carbon atoms and which is unsubstituted or is substituted by at least one substituent selected from the group consisting of a hydroxy group, a halogen atom, an alkoxy group having from 1 to 6 carbon atoms and an alkylthio group having from 1 to 6 carbon atoms; a cycloalkyl group having from 3 to 8 carbon atoms, an aryl group; or an aralkyl group; said aryl group having from 6 to 14 ring carbon atoms in a carbocyclic ring and are unsubstituted or are substituted by at least one substituent selected from the group consisting of substituents α and substituents β;
said aralkyl group are an alkyl group having from 1 to 6 carbon atoms and which are substituted by at least one aryl group as defined above;
said substituents α are selected from the group consisting of a hydroxy group, a halogen atom, an alkoxy group having from 1 to 6 carbon atoms and an alkylthio group having from 1 to 6 carbon atoms;
said substituents β are selected from the group consisting of an alkyl group which has from 1 to 6 carbon atoms and which is unsubstituted or are substituted by at least one substituent selected from the group consisting of a hydroxy group, a halogen atom, an alkoxy group having from 1 to 6 carbon atoms and an alkylthio group having from 1 to 6 carbon atoms; an alkanoyloxy group having from 1 to 6 carbon atoms; a mercapto group; an alkanoylthio group having from 1 to 6 carbon atoms; an alkylsulfinyl group having from 1 to 6 carbon atoms; a cycloalkloxy group having from 3 to 8 carbon atoms; a haloalkoxy group having from 1 to 6 carbon atoms; and an alkylenedioxy group having from 1 to 6 carbon atoms; or a pharmaceutically acceptable salt, solvate, or prodrug.

4. The method of claim 3 wherein:
R is a hydrogen atom, a halogen atom or an alkyl group having from 1 to 4 carbon atoms;
R¹ is a methyl group or an amino group;
R² is an unsubstituted phenyl group or a phenyl group which is substituted by at least one substituent selected from the group consisting of a halogen atom; an alkoxy group having from 1 to 4 carbon atoms; an alkylthio group having from 1 to 4 carbon atoms; an unsubstituted alkyl group having from 1 to 4 carbon atoms; an, alkyl group having from 1 to 4 carbon atoms and which is substituted by at least, one substituent selected from the group consisting of a halogen atom, an alkoxy group having from 1 to 4 carbon atoms and an alkylthio group having from 1 to 4 carbon atoms; a haloalkoxy group having from 1 to 4 carbon atoms; and an alkylenedioxy group having from 1 to 4 carbon atoms;
R³ is a hydrogen atom, a halogen atom, an unsubstituted alkyl group having from 1 to 4 carbon atoms or a substituted alkyl group having from 1 to 4 carbon atoms and substituted by at least one substituent selected from the group consisting of a halogen atom, an alkoxy group having from 1 to 4 carbon atoms and an alkylthio group having from 1 to 4 carbon atoms;
R⁴ is a hydrogen atom; an unsubstituted alkyl group having from 1 to 4 carbon atoms; a substituted alkyl group having from 1 to 4 carbon atoms and substituted by at least one substituent selected from the group consisting of a hydroxy group, a halogen atom, an alkoxy group having from 1 to 4 carbon atoms and an alkylthio group having from 1 to carbon atoms; a cycloalkyl group having from 3 to 6 carbon atoms; an aryl group which has from 6 to 10 ring carbon atoms and which is unsubstituted or is substituted by at least one substituent selected from the group consisting of a halogen atom; an alkoxy group having from 1 to 4 carbon atoms; an alkylthio group having from 1 to 4 carbon atoms; an unsubstituted alkyl group having from 1 to 4 carbon atoms; an alkyl group having from 1 to 4 carbon atoms and substituted by at least one substituent selected from the group consisting of a hydroxy group, a halogen atom, an alkoxy group having from 1 to 4 carbon atoms and an alkylthio group having from 1 to 4 carbon atoms; and a cycloalkyloxy group having from 3 to 7 carbon atoms; an aralkyl group having from 1 to 4 carbon atoms in the alkyl part and containing at least one said aryl group; or a pharmaceutically acceptable salt, solvate, or prodrug.

5. The method of claim 4 wherein:
R is a hydrogen atom;
R¹ is an amino group;
R² is an unsubstituted phenyl group or a phenyl group which is substituted by at least one substituent selected from the group consisting of a halogen atom, an alkoxy group having from 1 to 4 carbon atoms, an alkylthio group having from 1 to 4 carbon atoms, an alkyl group having from 1 to 4 carbon atoms, a haloalkyl group having from 1 to 4 carbon atoms, a haloalkoxy group having from 1 to 4 carbon atoms and a alkylenedioxy group having from 1 to 4 carbon atoms;
R³ is a hydrogen atom, a halogen atom, an alkyl group having from 1 to 4 carbon atoms or a haloalkyl group having from 1 to 4 carbon atoms;
R⁴ is a hydrogen atom; an unsubstituted alkyl group having from 1 to 4 carbon atoms; a substituted alkyl group having from 1 to 4 carbon atoms and substituted by at least one substituent selected from the group consisting of a hydroxy group and an alkoxy group having from 1 to 4 carbon atoms; a cycloalkyl group having from 3 to 6 carbon atoms; an aryl group which has from 6 to 10 ring carbon atoms and which is unsubstituted or is substituted by at least one substituent selected from the group consisting of a hydroxy group; a halogen atom; an alkoxy group having from 1 to 4 carbon atoms; an unsubstituted alkyl group having from 1 to 4 carbon atoms; an alkyl group having from 1 to 4 carbon atoms and which is unsubstituted or substituted by at least one halogen atom; and a cycloalkyloxy group having from 3 to 7 carbon atoms; and an aralkyl group having from 1 to 4 carbon atoms in the alkyl part and containing at least one said aryl group; or a pharmaceutically acceptable salt, solvate, or prodrug.

6. The method of claim 5 wherein the 1,2-diphenylpyrrole derivative is selected from the group consisting of: 4-methyl-2-(4-methylphenyl)-1-(4-sulfamoylphenyl)pyrrole; 2-(4-methoxyphenyl)-4-methyl-1-(4-sulfamoylphenyl)pyrrole; 2-(4-chlorophenyl)-4-methyl-1-(4-sulfamoylphenyl)pyrrole; 4-methyl-2-(4-methylthiophenyl)-1-(4-sulfamoylphenyl)pyrrole; 2-(4-ethoxyphenyl)-4-methyl-1-(4-sulfamoylphenyl)pyrrole; 2-(4-methoxy-3-methylphenyl)-4-methyl-1-(4-sulfamoylphenyl)pyrrole; 2-(3-fluoro-4-methoxyphenyl)-4-methyl-1-(4-sulfamoylphenyl)pyrrole; 2-(3,4-dimethylphenyl)-4-methyl-1-(4-sulfamoylphenyl)pyrrole; 4-methyl-1-(4-methylthiophenyl)-2-(4-sulfamoylphenyl)pyrrole; 1-(4-acetylaminosulfonylphenyl)-4-methyl-2-(4-methoxyphenyl)pyrrole; and 1-(4-acetylaminosulfonylphenyl)-4-methyl-2-(3,4-dimethylphenyl)pyrrole.

7. The method of claim 6 wherein the 1,2-diphenylpyrrole derivative is 2-(4-ethoxyphenyl)-4-methyl-1-(4-sulfamoylphenyl)-pyrrole.

8. The method of claim 2 wherein the inhibitor of both EGFR [ErbB1] and HER2 [ErbB2] has the following formula: 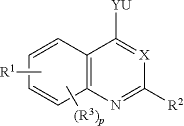
or a salt or solvate thereof;
wherein X is N or CH;
Y is a group W(CH₂), (CH₂)W, or W, in which W is O, S(O)ₘ wherein m is 0, 1, or 2, or NR^{a} wherein R^{a} is hydrogen or a C1-8 alkyl group;
R¹ represents a 5- or 6-membered heterocyclic ring containing 1 to 4 heteroatoms selected from N, O or S(O)ₘ, wherein m is as defined above, with the provisos that the ring does not have two adjacent O or S(O)ₘ atoms and that where the ring has only N as heteroatom(s) the ring is C-linked to the quinazoline or quinoline ring, R¹ is optionally substituted by one or more R³ groups;
each R³ is independently selected from the group consisting of amino, hydrogen, halogen, hydroxy, nitro, carboxy, formyl, cyano, trifluoromethyl, trifluoromethoxy, carbamoyl, ureido, guanidino, C1-8 alkyl, C1-8 alkoxy, C3-8 cycloalkoxyl, C4-8 alkylcycloalkoxy, C1-8 alkylcarbonyl, C1-8 alkoxycarbonyl, N—C1-4 allcylcarbamoyl, N,N-di-(C1-4 alkyl)carbamoyl, hydroxyamino, C1-4 alkoxyamino, C2-4 alkanoyloxyamino, C1-4 alkylamino, di(C1-4 alkyl)amino, di-(C1-4 alkyl)amino-C1-4 alkylene-(C1-4 alkyl)amino, C1-4 alkylamino-C1-4 alkylene-(C1-4 alkyl)amino, hydroxy-C1-4 alkylene-(C1-4 alkyl)amino, phenyl, phenoxy, 4-pyridon-1-yl, pyrrolidin-1-yl, imidazol-1-yl, piperidino, morpholino, thiomorpholino, thiomorpholino-1-oxide, thiomorpholino-1,1-dioxide, piperazin-1-yl, 4-C1-4 alkylpiperazin-1-yl, dioxolanyl, C1-8 alkylthio, arylthio, C1-4 alkylsulphinyl, C1-4 alkylsulphonyl, arylsulphonyl, arylsulphinyl, halogeno-C1-4 alkyl, hydroxy-C1-4 alkyl, C2-4 alkanoyloxy-C1-4 alkyl, C1-4 alkoxy-C1-4 alkyl, carboxy-C1-4 alkyl, formyl-C1-4 alkyl, C1-4 alkoxycarbonyl-C1-4-alkyl, carbamoyl-C1-4 alkyl, N—C1-4 alkylcarbamoyl-C1-4 alkyl, N,N-di-(C1-4 allcyl)carbamoyl-C1-4 alkyl, amino-C1-4 alkyl, C1-4 alkylamino-C1-4 alkyl, di-(C1-4 alkylamino-C1-4 alkyl, phenyl-C1-4 alkyl, 4-pyridon-1-yl-C1-4 alkyl, pyrrolidin-1-yl-C1-4 alkyl, imidazol-1-yl-C1-4 alkyl, piperidino-C1-4 alkyl, morpholino-C1-4 alkyl, thiomorpholino-C1-4 alkyl, thiomorpholino-1-oxide-C1-4 alkyl, thiomorpholino-1,1-dioxide-C1-4 alkyl, piperazin-1-yl-C1-4 alkyl, 4-C1-4 alkylpiperazin-1-yl-C1-4 alkyl, hydroxy-C2-4 alkoxy-C1-4 alkyl, C1-4 alkoxy-C2-4 alkoxy-C1-4 alkyl, hydroxy-C2-4 alkylamino-C1-4 alkyl, C1-4 alkoxy-C2-4 alkylamino-C1-4 alkyl, C1-4 alkylthio-C1-4 alkyl, C1-4 alkylsulphinyl-C1-4 alkyl, C1-4 alkylsulphonyl-C1-4 alkyl, hydroxy-C2-4 alkylthio-C1-4 alkyl, C1-4 alkoxy-C2-4 alkylthio-C1-4 alkyl, phenoxy-C1-4 alkyl, anilino-C1-4 alkyl, phenylthio-C1-4 alkyl, cyano-C1-4 alkyl, halogeno-C2-4 alkoxy, hydroxy-C2-4 alkoxy, C2-4 alkanoyloxy-C2-4 alkoxy, C1-4 alkoxy-C2-4 alkoxy, carboxy-C1-4 alkoxy, formyl-C1-4 alkoxy, C1-4 alkoxycarbonyl-C1-4 alkoxy, carbamoyl-C1-4 alkoxy, N—C1-4 alkylcarbamoyl-C1-4 alkoxy; N,N-di-(C1-4 alkyl)carbamoyl-C1-4 alkoxy, amino-C2-4 alkoxy, C1-4 alkylamino-C2-4 alkoxy, di-(C1-4 alkylamino-C2-4 alkoxy, di-(C1-4 alkyl-C2-4 alkoxy)amino-C2-4 alkoxy, C2-4 alkanoyloxy, hydroxy-C2-4 alkanoyloxy, C1-4 alkoxy-C2-4 alkanoyloxy, phenyl-C1-4 alkoxy, phenoxy-C2-4 alkoxy, anilino-C2-4 alkoxy, phenylthio-C2-4 alkoxy, 4-pyridon-1-yl-C2-4 alkoxy, piperidino-C2-4 alkoxy, morpholino-C2-4 alkoxy, thiomorpholino-C2-4 alkoxy, thiomorpholino-1-oxide-C2-4 alkoxy, thiomorpholino-1,1-dioxide-C2-4 alkoxy, piperazin-1-yl-C2-4 alkoxy, 4-C1-4 alkylpiperazin-1-yl-C2-4 alkoxy, pyrrolidin-1-yl-C2-4 alkoxy, imidazol-1-yl-C2-4 alkoxy, halogeno-C2-4 alkylamino, hydroxy-C2-4 alkylamino, C2-4 alkanoyloxy-C2-4 alkylamino, C1-4 alkoxy-C2-4 alkylamino, carboxy-C1-4 alkylamino, C1-4 alkoxycarbonyl-C1-4 alkylamino, carbamoyl-C1-4 alkylamino, N—C1-4 alkylcarbamoyl-C1-4 alkylamino, N,N-di-(C1-4 alkylcarbamoyl-C1-4 alkylamino, amino-C2-4 alkylamino, C1-4 alkylamino-C2-4 alkylamino, di-(C1-4 alkyl)amino-C2-4 alkylamino, phenyl-C1-4 alkylamino, phenoxy-C2-4 alkylamino, anilino-C2-4 alkylamino, 4-pyridon-1-yl-C2-4 alkylamino, pyrrolidin-1-yl-C2-4 alkylamino, imidazol-1-yl-C2-4 alkylamino, piperidino-C2-4 alkylamino, morpholino-C2-4 alkylamino, thiomorpholino-C2-4 alkylamino, thiomorpholino-1-oxide-C2-4 alkylamino, thiomorpholino-1,1-dioxide-C2-4 alkylamino, piperazin-1-yl-C2-4 alkylamino, 4-(C1-4 alkyl)piperazin-1-yl-C2-4 alkylamino, phenylthio-C2-4 alkylamino, C2-4 alkanoylamino, C1-4 alkoxycarbonylamino, C1-4 alkylsulphonylamino, C1-4 alkylsulphinylamino, benzamido, benzenesulphonamido, 3-phenylureido, 2-oxopyrrolidin-1-yl, 2,5-dioxopyrrolidin-1-yl, halogeno-C2-4 alkanoylamino, hydroxy-C2-4 alkanoylamino,hydroxy-C2-4 alkanoyl-(C1-4 alkyl)-amino, C1-4 alkoxy-C2-4 alkanoylamino, carboxy-C2-4 alkanoylamino, C1-4 alkoxycarbonyl-C2-4 alkanoylamino, carbamoyl-C2-4 alkanoylamino; N—C1-4 alkylcarbamoyl-C2-4 alkanoylamino, N,N-di-(C1-4 alkyl)carbamoyl-C2-4 alkanoylamino, amino-C2-4 alkanoylamino, C1-4 alkylamino-C2-4 alkanoylamino or di-(C1-4 alkyl)amino-C2-4 alkanoylamino; and
wherein said benzamido or benzenesulphonamido substitutent or any anilino, phenoxy or phenyl group on a R³ substituent may optionally have one or two halogeno, C1-4 alkyl or C1-4 alkoxy substituents; and wherein any substituent having a heterocyclic ring may optionally have one or two halogeno, C1-4 alkyl or C1-4 alkoxy substituents on said ring;
and wherein any substituent having a heterocyclic ring may optionally have one or two oxo or thioxo substituents on said ring;
or R³ represents a group selected from M1-M2-M3-M4, M1-M5 or M1-M2-M3′-M6
wherein
M1 represents a C1-4 alkyl group, wherein optionally a CH₂ group is replaced by a CO group;
M2 represents NR¹² or CR¹²R¹³, in which R¹² and R¹³ each independently represent H or C1-4 alkyl;
M3 represents a C1-4 alkyl group;
M3′ represents a C1-4 alkyl group or is absent;
M4 represents CN, NR¹²S(O)ₘR¹³, S(O)ₘNR¹⁴R¹⁵, CONR¹⁴R¹⁵, S(O)ₘR¹³ or CO₂R¹³, in which R¹², R¹³ and m are as above defined and R¹⁴ and R¹⁵ each independently represent H or C1-4 alkyl, or R¹⁴ and R¹⁵ together with the nitrogen atom to which they are attached form a 5- or 6-membered ring optionally containing 1 or 2 additional heteroatoms selected from N, O or S(O)ₘ in which ring any nitrogen atom present may optionally be substituted with a C1-4 alkyl group, and which ring may optionally contained one or two oxo or thioxo substituents;
M5 represents the group NR¹⁴R¹⁵, wherein R¹⁴ and R¹⁵ are as defined above, or M5 represents the group 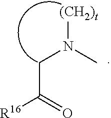
in which t represents 2 to 4 and R¹⁶ represents OH, OC1-4 alkyl or NR¹⁴R¹⁵; and
M6 represents a C3-6 cycloalkyl group, the group NR¹⁴R¹⁵, wherein R¹⁴ and R¹⁵ are as defined above, or a 5- or 6-membered heterocyclic ring system containing 1 to 4 heteroatoms selected from N, O or S;
and p is 0 to 3; or when p is 2 or 3, two adjacent R³ groups together form an optionally substituted methylenedioxy or ethylenedioxy group;
R² is selected from the group consisting of hydrogen, halogen, trifluoromethyl, C1-4 alkyl and C1-4 alkoxy;
U represents phenyl or a 5 to 10-membered mono or bicyclic ring system in which one or more of the carbon atoms is optionally replaced by a heteroatom independently selected from N, O and S(O)ₘ, wherein m is 0, 1 or 2, and wherein U is substituted by at least one independently selected R⁶ group and U is optionally substituted by at least one independently selected R⁴ group;
each R⁴ is independently hydrogen, hydroxy, halogen, C1-4 alkyl, C1-4 alkoxy, C1-4 alkylamino, di-[C1-4 alkyl]amino, C1-4 alkylthio, C1-4 alkylsulphinyl, C1-4 alkylsulphonyl, C1-4 alkylcarbonyl, C1-4 alkylcarbamoyl, di-[C1-4 alkyl]carbamoyl, carbamyl, C1-4 alkoxycarbonyl, cyano, nitro or trifluoromethyl;
each R⁶ is independently a group ZR⁷ wherein Z is joined to R⁷ through a (CH₂)ₚ group in which p is 0, 1 or 2 and Z represents a group V(CH₂), V(CF₂), (CH₂)V, (CF₂)V, V(CRR′), V(CHR) or V where R and R′ are each C1-4 alkyl and in which V is a hydrocarbyl group containing 0, 1 or 2 carbon atoms, carbonyl, dicarbonyl, CH(OH), CH(CN), sulphonamide, amide, O, S(O)ₘ or NR^{b} where R^{b} is hydrogen or R^{b} is C1-4 alkyl; and R⁷ is an optionally substituted C3-6 cycloalkyl; or an optionally substituted 5, 6, 7, 8, 9 or 10-membered carbocyclic or, heterocyclic moiety;
or R⁶ is a group ZR⁷ in which Z is NR^{b}, and NR^{b} and R⁷ together form an optionally substituted 5, 6, 7, 8, 9 or 10-membered carbocyclic or heterocyclic moiety.

9. The method of claim 8 wherein R¹ is a 5- or 6-membered heterocyclic ring substituted by one or more R³ groups selected from C1-4 alkylsulphinyl-C1-4 alkyl or C1-4 alkylsulphonyl-C1-4 alkyl.

10. The method of claim 9 wherein X represents N; Y represents NR^{a}, wherein R^{a} is hydrogen or C1-4 alkyl; R¹ represents furan, thiazole, thiophene, pyrrole, pyridine, pyrimidine, pyrazine, imidazole, oxazole, isoxazole, oxadiazole, tetrazole, triazole, dioxolane or a partially or fully hydrogenated derivative of any of these groups, optionally substituted with an R³ group selected from methylsulphonylethylaminomethyl, methylsulphonylethylamino-carbonyl, methylsulphinylethylamino-methyl, methylsulphinylethylamino-carbonyl, methylsulphonylpropylamino-methyl, methylsulphinylpropylamino-methyl, methylsulphonylpropyamino-carbonyl, methylsulphinylpropylamino-carbonyl, methylsulphonylethyl-(methylamino)-methyl, methylsulphonylethyl-(methylamino)-carbonyl, methylsulphinylethyl-(methylamino)-methyl, methylsulphinylethyl-(methylamino)-carbonyl, methylsulphonylpropyl-(methylamino)-methyl, methylsulphinylpropyl-(methylamino)-methyl, methylsulphonylpropyl-(methylamino)-carbonyl, methylsulphinylpropyl-(methylamino)-carbonyl, methylsulphonamidoethylamino-methyl, methylsulphonamidopropylamino-methyl, aminosulphonylethylaminomethyl, methylaminosulphonylethylaminomethyl, sarcosinamidomethyl, glycinylmethyl, glycinamidomethyl, glycinylmethyl methyl ester acetylaminoethylaminomethyl, piperazinylmethyl, methylpiperazinylmethyl, piperidinylmethyl, pyridylmethyl, N-(prolinamido)methyl, (N,N-dimethyl-prolinamido)methyl, pyridylaminomethyl, cyclopropylaminomethyl, N-(piperidin-4-yl)-N-methylaminomethyl, N,N-dimethylaminoprop-2-ylaminomethyl, N-(2-dimethylaminoethyl)-N-ethylaminomethyl, isopropylacetamido, N-morpholinylacetamido or tetrahydrofuranomethylaminomethyl and optionally further substituted by one or more C1-4 alkyl groups; p is 0; R² represents hydrogen; R⁴ represents hydrogen, halo or methyl; U represents phenyl, indolyl, benzimidazolyl or indazolyl; and R⁶ represents phenyl, benzyl, α-methylbenzyl, fluorobenzyl, difluorobenzyl, pyridylmethyl, benzenesulphonyl, phenoxy, fluorophenoxy, benzyloxy or fluorobenzyloxy.

11. The method of claim 10 wherein the inhibitor of both EGFR [ErbB1] and HER2 [ErbB2] is selected from the group:

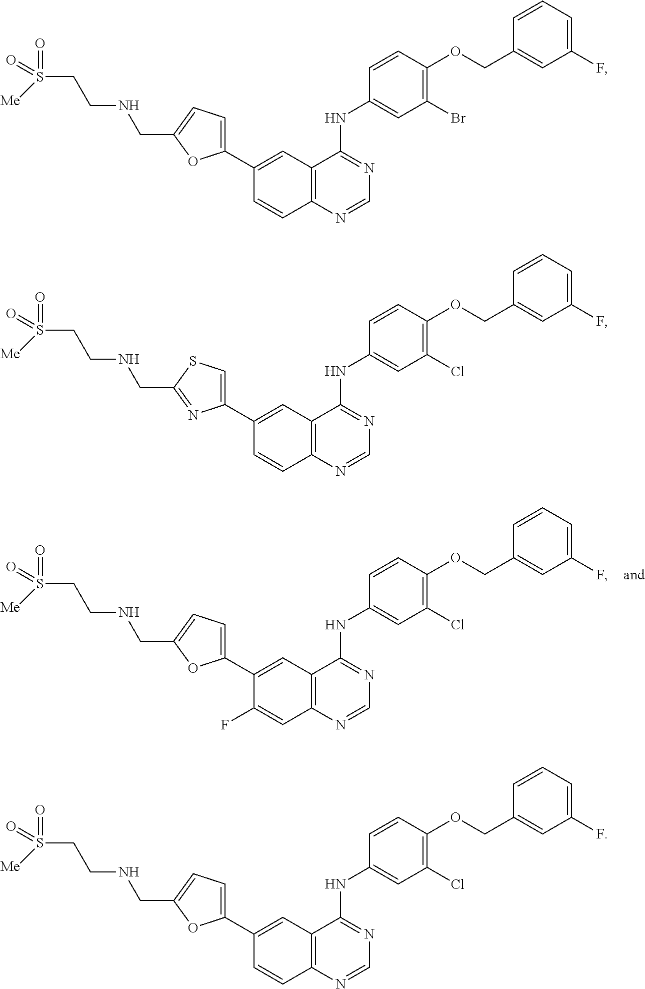

12. The method of claim 2 wherein the inhibitor of both EGFR [ErbB1] and HER2 [ErbB2] is lapatinib.

13. The method of claim 2 wherein the 1,2-diphenylpyrrole derivative is 2-(4-ethoxyphenyl)-4-methyl-1-(4-sulfamoylphenyl)-pyrrole and the inhibitor of both EGFR [ErbB1] and HER-2/n is lapatinib.

14. The method of claim 2 wherein the 1,2-diphenylpyrrole derivative and the inhibitor of both EGFR [ErbB1] and HER-2/n are administered sequentially in either order or simultaneously.

15. The method of claim 2 wherein the 1,2-diphenylpyrrole derivative is administered first.

16. The method of claim 2 wherein the inhibitor of both EGFR [ErbB1] and HER2 [ErbB2] is administered first.

17. The method of claim 2 further comprising administering to the subject one or more therapies in addition to the combination of a 1,2-diphenylpyrrole derivative and an inhibitor of both EGFR [ErbB1] and HER2 [ErbB2].

18. The method of claim 13 further comprising administering to the subject one or more therapies in addition to the combination comprising 2-(4-ethoxyphenyl)-4-methyl-1-(4-sulfamoylphenyl)-pyrrole and lapatinib.

19. The method of claim 13 further comprising administering to the subject capecitabine in addition to the combination comprising 2-(4-ethoxyphenyl)-4-methyl-1-(4-sulfamoylphenyl)-pyrrole and lapatinib.

20. The method of claim 19 comprising administering to the patient lapatinib at a dose ranging from about 500 mg/day to about 1500 mg/day, capecitabine at a dose ranging from about 1600 mg/m²/day to about 2500 mg/m²/day, and 2-(4-ethoxyphenyl)-4-methyl-1-(4-sulfamoylphenyl)pyrrole at a dose ranging, from about 100 mg/day to about 1200 mg/day continuously.

21.-123. (canceled)
